(19) United States
(12) Patent Application Publication
(10) Pub. No.: US 2012/0039840 A1
(43) Pub. Date: Feb. 16, 2012

(54) Use of IL-27-P28 to antagonize IL-6 mediated signaling

(76) Inventors: Christopher A. Hunter, Swarthmore, PA (US); Jason Scott Stumhofer, Plymouth Meeting, PA (US)

(21) Appl. No.: 12/998,799

(22) PCT Filed: Dec. 2, 2009
(86) PCT No.: PCT/US2009/006354

§ 371 Date: Oct. 24, 2011

Related U.S. Application Data
(60) Provisional application No. 61/200,727, filed on Dec. 2, 2008.

(51) Int. Cl. A61K 38/20; A61P 35/04; A61P 19/02; A61P 37/06; A61P 37/00

(52) U.S. Cl. 424/85.2

(57) ABSTRACT

Provided are methods increasing, or, alternatively, decreasing IL-6 and/or gp130-mediated signaling in mammalian subjects using p28. Methods of preventing and/or treating autoimmune disorders, cancer, transplant rejection, and other IL-6-associated diseases, as well as methods of enhancing an IL-6-mediated immune response, are also described.

### CROSS-REFERENCE TO RELATED APPLICATIONS

This application claims priority to and benefit of U.S. Provisional Patent Application Ser. No. 61/200,727, entitled, “USE OF IL-27-P28 TO ANTAGONIZE IL-6 MEDIATED SIGNALING”, by Christopher A. Hunter et al., filed on Dec. 2, 2008.

### STATEMENT AS TO RIGHTS TO INVENTIONS MADE UNDER FEDERALLY SPONSORED RESEARCH AND DEVELOPMENT

This invention was made with government support under Grant Nos. AI42334 from the National Institutes of Health. The government has certain rights to this invention.

### FIELD OF THE INVENTION

The invention relates to methods of suppressing an immune response in mammalian subjects using p28 to antagonize signaling through the IL-6 receptor. Alternatively, the invention provides methods of enhancing an IL-6 mediated immune response in such subjects by using a modulator that negatively regulates p28 activity. The invention provides evidence that p28 is an antagonist of IL-6-mediated signaling, which plays a role in inflammation, T-cell differentiation, and other immune responses. p28 can be useful in the treatment and/or prevention of conditions in which signaling through the IL6R or its downstream targets are implicated, e.g., cancer, inflammation, autoimmune disease, and others.

### BACKGROUND OF THE INVENTION

Cytokines are key immune molecules that are involved in driving the development of protective immunity but can also promote the development of inappropriate inflammation. Consequently, understanding the biology of cytokines can lead to therapies to either augment or reduce an immune response.

A number of recombinant cytokines are used in a variety of clinical settings. These include interleukin-2 (IL-2), GM-CSF, IL-11, IL-12 and type I interferons (IFNs). These proteins are primarily being used as stimulators of immune cells and to act as growth factors or to enhance anti-cancer or viral responses. Few cytokines have been used to inhibit the immune system. For example, inhibition has been attempted with IL-10, which works indirectly on accessory cell functions necessary for T cell functions and which was being developed specifically with Crohn's disease and Inflammatory Bowel Disease as targets (Herfarth et al. (2002) “IL-10 therapy in Crohn's disease: at the crossroads.” Gut 50: 146-147; Bhaysar et al. (2008) “Oral IL-10 gene delivery in a microsphere-based formulation for local transfection and therapeutic efficacy in inflammatory bowel disease” Gene Therapy 15: 1200-1209). Success with these has been limited.

Several companies have developed antibodies/antagonists specific for the cytokine TNF-α, which are currently used in the treatment of subjects with rheumatoid arthritis. This approach relies on the neutralization of endogenous cytokine to prevent inflammation. A similar approach has been pursued with antibodies specific for IL-1 and EL-6. One safety issue is that these treatments are associated with the development of opportunistic infections including TB and toxoplasmosis (Doan et al. (2005) “Rheumatoid Arthritis: An Overview of New and Emerging Therapies” J Clin Pharmacol 45: 751-762; Spadaro et al. (2009) “Monitoring Biological Therapies in Psoriatic Arthritis.” J Rheum 83: 69-70).

Antagonists of IL-12p40 have been tested in clinical trials for subjects with Crohn's disease and have now been approved for the treatment of psoriasis; antagonists of IL-15 are being tested for treatment of arthritis. In addition, an antagonist of the IL-1 receptor is being used to treat patients with rheumatoid arthritis to block the interaction of the pro-inflammatory cytokine IL-1 with its receptor.

The only cytokine being used to inhibit the immune system is type 1 IFNs for the treatment of multiple sclerosis. What is needed in the art is an additional way to inhibit the immune system, using naturally occurring cytokines or cytokine antagonists.

The products described above typically use antibodies to neutralize inflammatory cytokines. Because the antibodies are foreign proteins that typically induce an immune response their efficacy can be limited. An ideal drug would be a protein that is made naturally by the immune system that can antagonize cytokine function. For example, the IL-1 receptor antagonist (IL-1RA) is used to treat arthritis. However, this has limited efficacy and would benefit from combination with additional naturally occurring cytokine inhibitors.

The IL-27/IL-27R system has been previously studied in relation to suppression of the immune system. For example, Hunter et al., WO2004/069177, entitled “Methods for Modulating an Inflammatory Response,” described how the cytokine IL-27 can be used to inhibit an inflammatory response and explained how IL-27R is involved in controlling the intensity and duration of an immune response, and Hunter et al., WO 2008/011081, entitled “WSX-1/P28 as a Target for Anti-Inflammatory Responses” described how various IL-27/IL-27R complexes could be used to treat inflammation by inhibiting the T cell response through trans signaling. What is still needed however, is a better understanding of the role played by IL-27 and IL-27R and their various subunits in promoting inflammation. Furthermore, additional therapies are needed to more specifically control different types of inflammation. The present application provides data to further explain the role of the IL-27/IL-27R system in inflammatory responses and immune mediated disease and methods of treating such responses with a naturally occurring inhibitor of IL-6 signaling through the gp130 subunit of the IL-6 receptor (IL-6R).

### SUMMARY OF THE INVENTION

The present invention provides new methods of using p28 to treat or prevent conditions that are mediated via IL-6 signaling through gp130. This includes, but is not restricted to, inflammatory conditions, antibody-mediated conditions, coronary heart disease, cancer, angiogenesis, growth and development, and ischemic cerebrovascular disease. For example, in one aspect, a method of treating or preventing an autoimmune disease is provided. The method comprises administering p28 to a subject at risk for an autoimmune disease or to a subject who has an autoimmune disease. In some embodiments, the autoimmune disease is a B cell activated disease, e.g., it is mediated by B cell production of autoantibodies. For example, the autoimmune disease can be systemic lupus erythematosus (SLE), autoimmune hepatitis, bullous pemphigoid, celiac disease, Guillain-Barré syndrome (GBS), Goodpasture's syndrome, multiple sclerosis associated with the presence of autoantibodies to myelin basic protein (MBP) and myelin oligodendrocyte glycoprotein (MOG), pemphigus vulgaris, primary biliary cirrhosis, rheumatoid arthritis associated with rheumatoid factor, scleroderma, or Wegener's granulomatosis.

In one embodiment, the method further comprises administering an additional antagonist of T and B cell interactions with p28. Such additional antagonists include, but are not limited to those designed to block ICOS-ICOS-L, IL-21, or CD40-CD40L interactions.

In another embodiment, the p28 administered to the subject, e.g., a human, is a p28 variant, e.g., a p28 that has been modified to have an increased half-life or altered affinity for cytokine receptors.

In another aspect, a method of enhancing an IL-6 mediated immune response in a subject is provided. The method comprises administering an antagonist of p28, e.g., an inhibitor or negative modulator of p28 activity, to the subject, which subject, e.g., human patient is in need of immune enhancement, e.g., a recipient of a vaccination for infectious disease or immune mediated cancer therapy.

In another aspect, a method of treating or preventing a gp130-associated cancer is provided. The method comprises: identifying a subject who has or is predisposed to develop a gp130-associated cancer; and, administering p28 to the subject, e.g., without EB13. In some embodiments, the method further comprises administering an additional gp130 antagonist or other cytokine antagonist or other accepted anti-cancer agents.

In another aspect, a method of suppressing an immune response in a subject, comprising administering a combination of at least two inhibitors of Th17 differentiation, e.g., IL-1Ra and p28, to a subject is provided. Typically, one of the at least two inhibitors of Th17 differentiation is p28 and one of the at least two inhibitors of Th17 differentiation is an IL-1 antagonist, an IL-21 antagonist, a TNF antagonist, an IL-23 antagonist, or CTLA4-Ig.

In another aspect, a method of limiting transplant rejection comprising, administering p28 to a transplant recipient is provided. These and other aspects are described in more detail below.

In another aspect methods of detecting and measuring levels of p28 in a patient for diagnostic use are provided. In one embodiment, p28 levels can be used to determine the outcome or progress of a disease. In another embodiment, p28 levels can be used to distinguish which type of therapy would be most efficacious in a particular disease. If p28 levels are unusually high or low this information can be a useful indicator of what will happen next. For example, a Crohn's patient with high p28 levels might not be treated with an anti-IL-6 medication, but rather an anti-TNF. Alternatively, a similar patient that has low levels of p28 could be supplemented with p28 or another anti-IL-6 composition. The level of p28 becomes an indicator of which is the better treatment option. In another embodiment, high levels of p28 are associated with or indicative of different disease states. For example, a disease like SLE may be diagnosed based on a combination of criteria such as the presence of anti-nuclear antibodies as well as altered levels of p28. Also, the relative ratio of p28 to other cytokines (IL-12/IL-6) can be used to indicate disease state or treatment options.

It will be apparent to one of skill in the art that any of the methods and/or compositions provided by the invention can be used alone or in combination.

Kits that permit a practitioner to use the methods described herein, e.g., to monitor an IL-6 associated disease state, or to select a treatment and/or determine a prognosis for an IL-6 associated disease in a subject are also a feature of this invention. The kits can include a recombinant p28, a p28 variant, and/or p28 antagonists (e.g., p28 inhibitors or negative modulators of a p28 activity). The kits can optionally contain recombinant constructs comprising genes encoding, e.g., p28 or other p28-associated signaling components, and/or the like. The kits can also include additional useful reagents, such as antibodies, buffers, and the like. Such kits also typically include, e.g., instructions for use of the compounds and other reagents, e.g., to practice the methods of the invention, as well as any packaging materials for packaging the components of the kits.

### BRIEF DESCRIPTION OF THE DRAWINGS

FIG. 1 is a schematic illustration of IL-6 mediated signaling and inhibition of that signaling by p28.

FIG. 2. Bone marrow derived dendritic cells were stimulated in vitro with LPS alone or in combination with p28 for 24 hours. The levels of RANTES and IL-1beta were measured using an assay system provided by Rules Based Medicine.

FIG. 3 provides data for bone marrow derived macrophages from C57BI/6 mice that were cultured for 24 hours in the presence of media, IL-6 or recombinant p28. Levels of RANTES mRNA were measured by real time PCR. In a dose dependent fashion IL-6 induces RANTES but this effect is antagonized in the presence of p28.

FIG. 4 provides flow cytometry data showing biological activity of p28 in vitro in CD4 and CD8 cells. Flow cytometry of CD4+ T cells and CD8+ T cells isolated from C57BL/6 mice and activated with anti-CD3 and anti-CD28 in TH-17-inducing conditions in the presence or absence of IL-27 (a-c) or p28 (c). CD4+ and CD8+ T cells cultured for 4 d and 3 d, respectively, were stimulated for 4 h with PMA and ionomycin in the presence of brefeldin A before being stained for intracellular IL-17 or IFN-gamma. Plots are gated on CD4+ or CD8+ T cells; numbers in quadrants represent the frequency of cells in each. Data are representative of three independent experiments.

FIG. 5 shows that that p28 inhibits phosphorylation of STAT1 in response to IL-6.Splenocytes from B 6 mice were incubated with these factors and flow cytometry was used to measure levels of STAT activation. In resting cells, <5% of the cells contain phosphorylated STAT 1. The data presented show a 5 and 15 min time point, but this effect persisted over several hours.

FIG. 6 shows that p28 antagonizes IL-6-mediated phosphorylation of STAT3 and STAT1.Splenocytes from B6 mice were incubated with IL-6 alone or in combination with p28 and flow cytometry was used to measure levels of STAT activation. In resting cells, <5% of the cells contain phosphorylated STAT 1 or 3. The data presented show a single 15 min time point, but this effect persisted over several hours.

FIG. 7 (Panels A-D) illustrates regulation of IL-27 p28 gene expression in macrophages and dendritic cells.

FIG. 8 (Panels A and B) indicates that IL-6 and IL-27 induced high levels of STAT1 and STAT3 phosphorylation in purified CD4⁺ T cells.

FIG. 9: Panel A shows a construct that was used in making p28 transgenic mice. Panels B through D show that low basal levels of p28 were detected in the serum of naïve wild-type mice, but the level of IL-27p28 measured in the serum of naïve p28Tg mice was significantly higher than their wild-type littermates. Panels C, D, and E provide flow cytometry data for p28 wild type and p28Tg in vitro in CD4 cells. Panel C shows data for IL-17, Panel D for IL-10 and Panel E for PSTAT1.

FIG. 10 Panel A shows that the p28Tg mice lack B-la B cells in the peritoneum and Panel B indicates that the transgenic p28Tg mice show no developmental defects by illustrating various stages of B-2 B cell development observed within the bone marrow and spleen of p28Tg mice compared to their wild-type littermates.

FIG. 11 (Panels A and B) illustrate number of IgG and IgM antibody-secreting cells in lymphoid compartments of naïve and p28Tg mice.

FIG. 12 (Panels A and B): Panel A provides data for production of IL-17 by the transgenic compared to the wild type control mice; Panel B provides flow cytometry data illustrating production of IL-10 and phosphorylation of STAT1.

FIG. 13 (Panels A-D) provides data indicating that p28Tg mice are unable to form GC reactions, which are necessary for B cell class switching and affinity maturation to occur in response to immunization with the T cell dependent antigen NP-CGG.

FIG. 14 (Panels and B) provides data for GC reactions at day 14 post-immunization showing that the wild-type mice were able to form distinct GCs in the spleen as assessed by the GC marker peanut agglutinin (PNA) and CD3 staining, while the p28Tg mice failed to generate GCs at all, with the majority of the PNA⁺ B cells primarily residing outside of the follicle (Panel A).

FIG. 15 shows the results of experiments that were performed to determine the effects of p28 overexpression in splenocytes on IL-12, IL-10, and IFNγ production.

FIG. 16 shows the results of experiments performed to determine IFNγ levels in various tissues in p28 transgenic mice.

### DEFINITIONS

Unless defined otherwise, all technical and scientific terms used herein have the same meaning as commonly understood by one of ordinary skill in the art to which the invention pertains. The following definitions supplement those in the art and are directed to the current application and are not to be imputed to any related or unrelated case, e.g., to any commonly owned patent or application. Although any methods and materials similar or equivalent to those described herein can be used in the practice of or testing of the present invention, the preferred materials and methods are described herein. Accordingly, the terminology used herein is for the purpose of describing particular embodiments only, and is not intended to be limiting.

As used in this specification and the appended claims, the singular forms “a,” “an” and “the” include plural referents unless the context clearly dictates otherwise. Thus, for example, reference to “a cytokine” includes a plurality of cytokines; reference to “a cell” includes mixtures of cells, and the like.

“Interleukin-27” or “IL-27” is a heterodimeric cytokine that includes “EBI3” and “p28.” Other names for p28 in the literature include interleukin 30 or IL30. p28 is described, for example, in entry 608273 in the Online Mendelian Inheritance in Man database, on the world wide web at www (dot) ncbi (dot) nlm (dot) nih (dot) gov/Omim. See also protein sequence id NP_{—}663634 and NP_{—}663611.1, nucleotide sequence accession number NM_{—}145659 and NM_{—}145636.1, and Gene ID 246778 and 246779, available, e.g., through the National Center for Biotechnology Information's Entrez protein, nucleotide, and gene browsers on the world wide web at www (dot) ncbi (dot) nlm (dot) nih (dot) gov/entrez. EBI3 (“Epstein-Barr virus-induced gene 3”) is described, for example, in entry 605816 in the Online Mendelian Inheritance in Man database. See also protein sequence id NP_{—}005746 and NP_{—}056581.1, nucleotide sequence accession number NM_{—}005755 and NM_{—}015766, and Gene ID 10148 and 50498.

IL-27 signals through a receptor complex that includes the class I cytokine receptors “WSX-1” and “gp130.” Other names for WSX-1 in the literature include T-cell cytokine receptor (TCCR), interleukin 27 receptor alpha (IL27RA), and interleukin 27 receptor (IL27R). WSX-1 is described, for example, in entry 605350 in the Online Mendelian Inheritance in Man database. See also protein sequence id NP_{—}004834 and NP_{—}057880.1, nucleotide sequence accession number NM_{—}004843 and NM_{—}016671, and Gene ID 9466 and 50931. Other names for gp130 in the literature include interleukin 6 signal transducer (IL6ST). gp130 is described, for example, in entry 600694 in the Online Mendelian Inheritance in Man database. See also protein sequence id NP_{—}002175 and NP_{—}034690, nucleotide sequence accession number NM_{—}002184 and NM_{—}010560, and Gene ID 3572 and 16195.

A “p28 polypeptide” (or, analogously, “gp130 polypeptide” or “EBI3 polypeptide”) refers to a polypeptide including the full-length amino acid sequence of a naturally occurring p28 or a subsequence or fragment thereof, or a variant thereof (i.e., a variant of the full-length sequence or the subsequence). p28 polypeptides also include polypeptides homologous or substantially identical thereto, and subsequences or variants thereof. As described above, p28 is one of the heterodimeric subunits comprising IL-27.

An “agonist” is a compound (e.g., an endogenous substance or a drug) that can bind to and activate a receptor, thereby initiating a response (e.g., a physiological or pharmacological response) characteristic of that receptor. Agonists can be, e.g., full agonists or partial agonists.

An “antagonist” is a compound (e.g., a drug) that can bind to a receptor and prevent an agonist from binding to and activating that receptor. Typically, binding of an antagonist to a receptor forms a complex that does not give rise to any response, as if the receptor were unoccupied. Alternatively, the antagonist can be a partial agonist.

It is worth noting that certain compounds can be classified as both an agonist and an antagonist. For example, a “mixed agonist-antagonist” (also called a “partial agonist”) is a compound which possesses affinity for a receptor, but which, unlike a full agonist, will elicit only a small degree of the response characteristic of that receptor, even if a high proportion of receptors are occupied by the compound. Such occupancy of the receptors by the partial agonist can prevent binding of a full agonist (e.g., an endogenous agonist) to the receptor.

The term “inflammatory condition” refers to any disease, disorder, or other condition in which inflammation is present. The inflammation can be, e.g., acute, chronic, localized, and/or systemic and can be mediated by cells of the innate and/or adaptive immune response.

An “anti-inflammatory” composition is one that ameliorates inflammation. For example, the composition can cause full or partial resolution of or prevent further worsening of an inflammatory condition.

A “subject” herein is typically a human, but can be a non-human mammal. Exemplary non-human mammals include laboratory, domestic, pet, sport, and stock animals, e.g., mice, cats, dogs, horses, and cows. In one aspect, a subject is eligible for treatment of an inflammatory condition. For the purposes herein, such eligible subject is one that is experiencing or has experienced one or more signs, symptoms, or other indicators of the inflammatory condition. Diagnosis of the condition (and determination of eligibility for treatment) can be performed as established in the art.

“Treatment” of a subject herein refers to both therapeutic treatment and prophylactic or preventative measures. Those in need of treatment include those already with an inflammatory condition as well as those in which inflammation is to be prevented. Hence, the subject may have been diagnosed as having an inflammatory condition or may be predisposed or susceptible to an inflammatory condition.

The term “ameliorates” or “amelioration” as used herein refers to a decrease, reduction or elimination of a condition, disease, disorder, or phenotype, including an abnormality or symptom.

A “symptom” of a condition, disease or disorder is any morbid phenomenon or departure from the normal in structure, function, or sensation, experienced by a subject and indicative of the condition, disease or disorder.

The expression “therapeutically effective amount” refers to an amount that is effective for preventing, ameliorating, or treating a condition, disease or disorder. For example, a “therapeutically effective amount” of a polypeptide or complex refers to an amount of the polypeptide or complex that is effective for preventing, ameliorating, or treating the specified inflammatory condition. Similarly, a “therapeutically effective amount” of a combination of a polypeptide or complex and a second compound (e.g., an antibody, another polypeptide or complex, or a drug) refers to an amount of the polypeptide or complex and an amount of the second compound that, in combination, are effective for preventing, ameliorating, or treating the specified condition.

It is to be understood that the terminology “a combination of two compounds does not mean that the compounds have to be administered in admixture with each other. Thus, treatment with or use of such a combination encompasses a mixture of the compounds or separate administration of the compounds, and includes administration on the same day or different days. Thus the terminology “combination” means two or more compounds are used for the treatment, either individually or in admixture with each other. When a polypeptide or complex and a second compound, for example, are administered in combination to a subject, the polypeptide or complex is present in the subject at a time when the second compound is also present in the subject, whether the polypeptide or complex and second compound are administered individually or in admixture to the subject.

The term “isolated” refers to a biological material, such as a nucleic acid or a polypeptide, which is substantially free from components that normally accompany or interact with it in its naturally occurring environment. The isolated material optionally comprises material not found with the material in its natural environment, e.g., a cell. For example, if the material is in its natural environment, such as a cell, the material has been placed at a location in the cell (e.g., genome or genetic element) not native to a material found in that environment. For example, a naturally occurring nucleic acid (e.g., a coding sequence, a promoter, an enhancer, etc.) becomes isolated if it is introduced by non-naturally occurring means to a locus of the genome (e.g., a vector, such as a plasmid or virus vector, or amplicon) not native to that nucleic acid. Such nucleic acids are also referred to as “heterologous” nucleic acids. An isolated polypeptide, for example, is in an environment (e.g., a cell culture system, or purified from cell culture) other than the native environment of wild-type polypeptide. Preferably, the isolated polypeptide is substantially free from proteins or polypeptides or other contaminants that are found in its natural environment that would interfere with its therapeutic, diagnostic, prophylactic, research or other use.

The term “recombinant” indicates that the material (e.g., a nucleic acid or a polypeptide) has been artificially or synthetically (non-naturally) altered by human intervention. The alteration can be performed on the material within, or removed from, its natural environment or state. For example, a “recombinant nucleic acid” is one that is made by recombining nucleic acids, e.g., during cloning, DNA shuffling or other procedures; a “recombinant polypeptide” or “recombinant protein” is, e.g., a polypeptide or protein that is produced by expression of a recombinant nucleic acid.

The term “nucleic acid” encompasses any physical string of monomer units that can be corresponded to a string of nucleotides, including a polymer of nucleotides (e.g., a typical DNA or RNA polymer), PNAs, modified oligonucleotides (e.g., oligonucleotides comprising nucleotides that are not typical to biological RNA or DNA, such as 2′-O-methylated oligonucleotides), and the like. A nucleic acid can be e.g., single-stranded or double-stranded. Unless otherwise indicated, a particular nucleic acid sequence of this invention encompasses complementary sequences, in addition to the sequence explicitly indicated.

A “polynucleotide sequence” or “nucleotide sequence” is a polymer of nucleotides (an oligonucleotide, a DNA, a nucleic acid, etc.) or a character string representing a nucleotide polymer, depending on context. From any specified polynucleotide sequence, either the given nucleic acid or the complementary polynucleotide sequence (e.g., the complementary nucleic acid) can be determined.

“Expression of a gene” or “expression of a nucleic acid” means transcription of DNA into RNA (optionally including modification of the RNA, e.g., splicing), translation of RNA into a polypeptide (possibly including subsequent modification of the polypeptide, e.g., posttranslational modification), or both transcription and translation, as indicated by the context.

The term “gene” is used broadly to refer to any nucleic acid associated with a biological function. Genes typically include coding sequences and/or the regulatory sequences required for expression of such coding sequences. The term “gene” applies to a specific genomic sequence, as well as to a cDNA or an mRNA encoded by that genomic sequence. Genes also include non-expressed nucleic acid segments that, for example, form recognition sequences for other proteins. Non-expressed regulatory sequences include “promoters” and “enhancers,” to which regulatory proteins such as transcription factors bind, resulting in transcription of adjacent or nearby sequences. A “tissue specific” promoter or enhancer is one that regulates transcription in a specific tissue type or cell type, or types.

An “expression vector” is a vector, such as a plasmid, which is capable of promoting expression as well as replication of a nucleic acid incorporated therein. Typically, the nucleic acid to be expressed is “operably linked” to a promoter and/or enhancer, and is subject to transcription regulatory control by the promoter and/or enhancer.

As used herein, the term “encode” refers to any process whereby the information in a polymeric macromolecule or sequence string is used to direct the production of a second molecule or sequence string that is different from the first molecule or sequence string. As used herein, the term is used broadly, and can have a variety of applications. In one aspect, the term encode describes the process of semi-conservative DNA replication, where one strand of a double-stranded DNA molecule is used as a template to encode a newly synthesized complementary sister strand by a DNA-dependent DNA polymerase. In another aspect, the term encode refers to any process whereby the information in one molecule is used to direct the production of a second molecule that has a different chemical nature from the first molecule. For example, a DNA molecule can encode an RNA molecule (e.g., by the process of transcription incorporating a DNA-dependent RNA polymerase enzyme). Also, an RNA molecule can encode a polypeptide, as in the process of translation. In another aspect, a DNA molecule can encode a polypeptide, where it is understood that “encode” as used in that case incorporates both the processes of transcription and translation.

A “polypeptide” is a polymer comprising two or more amino acid residues (e.g., a peptide or a protein). The polymer can additionally comprise non-amino acid elements such as labels, quenchers, blocking groups, or the like and can optionally comprise modifications such as glycosylation or the like. The amino acid residues of the polypeptide can be natural or non-natural and can be unsubstituted, unmodified, substituted or modified.

An “amino acid sequence” is a polymer of amino acid residues (a protein, polypeptide, etc.) or a character string representing an amino acid polymer, depending on context.

A “subsequence” or “fragment” is any portion of an entire sequence, up to and including the complete sequence. Typically a subsequence or fragment comprises less than the full-length sequence. Optionally, and depending on the length of the complete sequence, a subsequence can include, e.g., at least about 25, at least about 50, at least about 75, at least about 100, at least about 200, at least about 300, or at least about 500 contiguous amino acids of the complete sequence.

The term “variant” (or “derivative”) with respect to a polypeptide indicates the variant has an amino acid sequence that is altered by one or more amino acids with respect to a reference sequence (e.g., a naturally occurring sequence, e.g., a naturally occurring p28 amino acid sequence). The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variation can also include amino acid deletion or insertion, or both. Guidance in determining which amino acid residues can be substituted, inserted, or deleted without eliminating biological or immunological activity can be found using computer programs well known in the art, for example, DNASTAR software. Examples of conservative substitutions are also described below. Variants also include fusion proteins and polypeptides otherwise derived from the polypeptide. Optionally, the variant is at least about 60% identical to the reference sequence (e.g., a naturally occurring sequence, e.g., a human or mouse p28 polypeptide sequence) or a subsequence thereof. Frequently, such sequences are at least about 70%, at least about 75%, at least about 80%, at least about 85%, at least about 90%, at least about 95%, at least about 98%, at least about 99%, or at least about 99.5% identical to the reference sequence, for example, over a subsequence of the reference sequence including, e.g., at least about 25, at least about 50, at least about 75, at least about 100, at least about 200, at least about 300, or at least about 500 contiguous amino acids of the reference sequence.

The term “derived from” refers to a component that is isolated from or made using a specified molecule, or information from the specified molecule. For example, a polypeptide that is derived from a second polypeptide can include an amino acid sequence or subsequence that is identical or substantially identical to the amino acid sequence or subsequence of the second polypeptide. In the case of polypeptides, the derived species can be obtained by, for example, naturally occurring mutagenesis, artificial directed mutagenesis, artificial random mutagenesis, or other techniques for producing recombinant polypeptides. Mutagenesis of a polypeptide typically entails manipulation of the polynucleotide that encodes the polypeptide.

The term “fusion protein” indicates that the protein includes polypeptide components derived from more than one parental protein or polypeptide. Typically, a fusion protein is expressed from a fusion gene in which a nucleotide sequence encoding a polypeptide sequence from one protein is appended in frame with, and optionally separated by a linker from, a nucleotide sequence encoding a polypeptide sequence from a different protein. The fusion gene can then be expressed by a cell (or in an in vitro expression system) as a single recombinant fusion protein. As another example, a fusion protein can be produced by covalently connecting (e.g., in vitro) the polypeptide components after each component is produced separately.

A “domain” of a protein is any portion of the entire protein, up to and including the complete protein but typically comprising less than the complete protein. A domain can, but need not, fold independently of the rest of the protein chain and/or be correlated with a particular biological function or location (e.g., a ligand binding domain, or a cytosolic, transmembrane or extracellular domain).

An “activity modulator” modulates (enhances or inhibits) an activity of a polypeptide or complex (e.g., a receptor or receptor ligand), either partially or completely. A modulator can be, e.g., a small molecule, a polypeptide, an antibody, a nucleic acid, etc.

As used herein, “mediated” refers to an effect relating to or being the part of immunity or the immune response that is conveyed through an intermediate agent or signaling mechanism that can optionally include, e.g., STAT3, ras, JAKs, SHC, and/or the like, For example, Th17 T cell differentiation can result from of IL-6 mediated signaling.

As used herein, “IL-6 mediated signaling” refers to the signal transduction cascade initiated by the binding of IL-6 (e.g., also known in the literature as BSF2, HSF, and IFNB2) to an IL-6 receptor (e.g., IL-6R, which is a transmembrane heterodimer comprising IL6-Rα and p130). For example, IL-6-mediated signaling by, e.g., T cells and macrophages, stimulates Th 17 development, inflammation, B cell proliferation, and B cell class switching.

As used herein, an “autoimmune disease” refers to any of a large group of diseases characterized by abnormal functioning of the immune system that causes a host's immune system to produce antibodies against its own tissues. Autoimmune diseases arise from an aberrant immune response of the body against substances and tissues normally present in the body. Examples include, e.g., Systemic lupus erythematosus (SLE), Autoimmune hepatitis, Bullous pemphigoid, Celiac disease, Guillain-Barré syndrome (GBS), Goodpasture's syndrome, Multiple sclerosis associated with the presence of autoantibodies to Myelin basic protein (MBP) and Myelin oligodendrocyte glycoprotein (MOG), Pemphigus Vulgaris, Primary biliary cirrhosis, Rheumatoid arthritis associated with rheumatoid factor, Scleroderma, or Wegener's granulomatosis.

A “B cell mediated autoimmune disease” is an autoimmune disease that results from a loss of “self” tolerance that is almost entirely restricted to the autoantibody responses produced by B lymphocytes.

The term “inhibition of Th17 differentiation” is used herein to refer to an environment comprising a cytokine millieu that suppresses the development of Th17 cells, i.e., a class of T cells that produce interleukin 17. Th 17 cells are developmentally distinct from Th1 and Th2 cells and are thought to play a role in inflammation and severe autoimmune diseases.

A variety of additional terms are defined or otherwise characterized herein.

### DETAILED DESCRIPTION

The cytokines IL-6, IL-12, IL-23 and IL-27 are closely related to one another based on similarities of their structural motifs, a common four-helix bundle, and their shared usage of various receptor subunits. These type I cytokines initiate their activity through membrane bound receptor complexes that include either gp130 or IL-12Rβ1 in order to influence the development and regulation of inflammatory responses (Kastelein et al. (2007) “Discovery and biology of IL-23 and IL-27: related but functionally distinct regulators of inflammation.” Annu Rev Immunol 25: 221-242). These cytokines have received a lot of recent attention due to their ability to direct T_{H}1 and T_{H}17 responses as well as the ability of IL-27 to regulate these responses. IL-6, the prototypical member of this family is a single subunit cytokine that binds to gp130 and a unique surface bound IL-6Rα chain. In addition, the IL-6Rα chain can be secreted as a soluble version due to proteolytic cleavage by the metalloproteinase ADAM17 or translation of an alternatively spliced mRNA (Briso et al. (2008) “Cutting edge: soluble IL-6R is produced by IL-6R ectodomain shedding in activated CD4 T cells.” J Immunol 180: 7102-7106; Jones et al. (2001) “The soluble interleukin 6 receptor: mechanisms of production and implications in disease.” FASEB J 15: 43-58; Matthews et al. (2003) “Cellular cholesterol depletion triggers shedding of the human interleukin-6 receptor by ADAM10 and ADAM17 (TACE).” J Biol Chem 278: 38829-38839). The sIL-6R (e.g., soluble IL-6R) can form a complex with IL-6, which can then bind gp130 and transduce a signal through a process termed trans-signaling (Jones (2005) “Directing transition from innate to acquired immunity: defining a role for IL-6.” J Immunol 175: 3463-3468; Jones et al. (2005) “IL-6 transsignaling: the in vivo consequences.” J Interferon Cytokine Res 25: 241-253). Recent reports have indicated that this latter process has been implicated in the control of leukocyte recruitment, activation and apoptotic clearance in a number of chronic inflammatory diseases such as inflammatory bowel disease, peritonitis, rheumatoid arthritis and asthma (Jones (2005) “Directing transition from innate to acquired immunity: defining a role for IL-6.” J Immunol 175: 3463-3468; Jones et al. (2005) “IL-6 transsignaling: the in vivo consequences.” J Interferon Cytokine Res 25: 241-253).

IL-27, a member of the type I cytokine family discussed above, is a heterodimeric cytokine composed of p28 and EBI3 (Pflanz et al. (2002) “IL-27, a heterodimeric cytokine composed of EBI3 and p28 protein, induces proliferation of naive CD4(+) T cells.” Immunity 16: 779-790). While p28 is a four-helix bundle protein similar to IL-6 the structure of EBI3 resembles that of the sIL-6R. Unlike IL-6, IL-27 employs a unique receptor subunit IL-27ra (also known as WSX-1 or TCCR) to pair with gp130 for signaling (Pflanz et al. (2002) “IL-27, a heterodimeric cytokine composed of EBI3 and p28 protein, induces proliferation of naive CD4(+) T cells.” Immunity 16: 779-790; Pflanz et al. (2004) “WSX-1 and glycoprotein 130 constitute a signal-transducing receptor for IL-27.” J Immunol 172: 2225-2231). Whereas a disulfide bond links the individual subunits of the other heterodimeric cytokines of this family, IL-12 and IL-23, the subunits of IL-27 do not interact in this manner suggesting an alternative mechanism of folding and assembly for IL-27 (Batten et al. (2007) “The biology and therapeutic potential of interleukin 27.” J Mol Med 85: 661-672). The current model for expression of these heterodimeric cytokines dictates that their secretion is dependent on the transcription of the smaller subunit proteins: IL-12p35, IL-23p19 and IL-27p28, as these loci are tightly regulated as compared to their receptor-like subunit counterparts, p40 and EBI3, which show a constitutive pattern of expression in antigen presenting cells. Furthermore, this difference in transcriptional regulation can result in the secretion of individual subunits of these cytokines. For example, the p40 subunit is produced in greater abundance than its partner p35, which results in the formation of p40 homodimers that can function as natural antagonists of IL-12 signaling (Heinzel et al. (1997) “In vivo production and function of IL-12 p40 homodimers.” J Immunol 158: 4381-4388), and have been assigned chemotactic properties (Khader et al. (2006) “Interleukin 12p40 is required for dendritic cell migration and T cell priming after Mycobacterium tuberculosis infection.” J Exp Med 203: 1805-1815). Thus, it is possible that the p28 and EBI3 subunits of IL-27 can be secreted independently from the other, thus allowing for extracellular association or pairing of each subunit with itself or other proteins. While there has been no reported evidence that indicates p28 or EBI3 form homodimers, Pflanz et al. determined that murine p28 can be secreted independently of EBI3, but no functional role for p28 was found in a number of bioassays (Pflanz et al. (2002) “IL-27, a heterodirneric cytokine composed of EBI3 and p28 protein, induces proliferation of naive CD4(+) T cells.” Immunity 16: 779-790). Yet, previous work from this laboratory has shown that purified p28 was capable of suppressing IL-17 production by CD4⁺ T cells grown under Th17 polarizing conditions in vitro suggesting that p28 has biological activity, or that EBI3 was present in the culture conditions to form heterodimers (Stumhofer et al. (2006) “Interleukin 27 negatively regulates the development of interleukin 17-producing T helper cells during chronic inflammation of the central nervous system.” Nat Immunol 7: 937-945).

While IL-27 by itself or in synergy with TGF-β has been found to promote IL-10 production by CD4+ T cells (Stumhofer et al. (2007) “Interleukins 27 and 6 induce STAT3-mediated T cell production of interleukin 10.” Nat Immunol 8: 1363-1371), p28 alone or in the presence of TGF-β did not support the development of IL-10 producing T cells. Thus, these results indicate that p28 does not possess the same biological activity as IL-27 in these in vitro assays.

However, new data presented herein indicates that p28 can antagonize pro-inflammatory responses through blocking IL-6 mediated signaling. In other words, p28 does not send an inhibitory signal directly to the T cells but rather blocks the ability of another cytokine to send a positive signal. The p28 subunit of IL-27 alone, e.g., in the absence of EBI3, uses a pathway distinct from that of the heterodimeric IL-27 to antagonize the ability of IL-6 to promote T cell macrophage and dendritic cell responses. These findings suggest that p28 alone can serve as an antagonist of gp130 signaling, and can be useful as an inhibitor of cytokine signaling, e.g., IL-6 mediated signaling, through this receptor subunit. Evidence from the example below suggests that this activity does not require EBI3. Therefore, p28 or a modified version thereof (i.e., a p28 variant) can be used to treat inflammation, either by administration of a p28 compound to a subject or by increasing expression of p28 in a subject. This evidence provides novel treatments and the ability to tailor a treatment to the type of inflammatory response a subject is experiencing or is likely to experience.

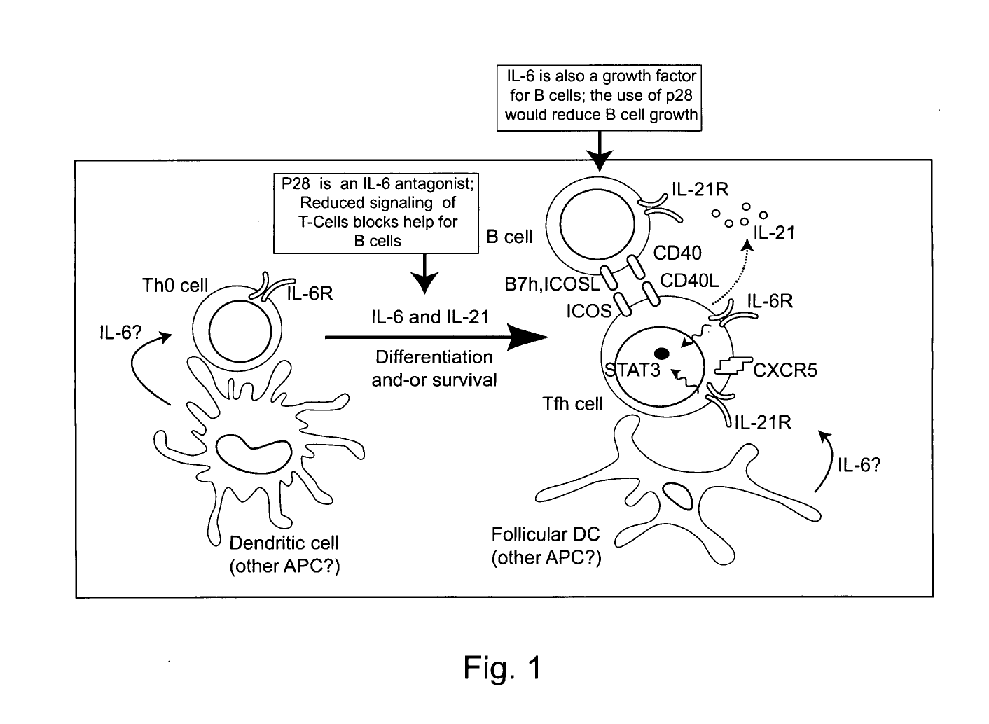

As shown in FIG. 1, secretion of IL-6 by dendritic cells and/or by other antigen presenting cells (APCs) stimulates the differentiation of naïve CD4⁺ T cells into Tfh cells by initiating cellular events including activation of JAK kinases and activation of ras-mediated signaling. Tfh cells are a unique subset of CD4⁺ T cells that produce the proinflammatory cytokines Il-17A, Il-17F, Il-17A/F heterodimer and Il-21. The secretion of IL-21 by Tfh cells induces CD40L-stimulated and ICOS-stimulated naïve antigen-primed B cells to undergo class switching recombination to acquire expression of IgG and IgA, and secrete large amounts of IgG, IgA and IgE. Isotype switching induced in ICOS- and CD40L-stimulated naïve B cells by IL-21 has also been linked to cell division (i.e., B cell proliferation) (Tangye et al. (2007) “Follicular CD4+ T helper cells induce human B cells to undergo Ig isotype switching and differentiation to Ig-secreting cells through the production of IL-21.” J Immunol 178: 95.1). As provided herein, the use of p28, which downregulates IL-6 mediated signaling, can reduce inflammation, B cell growth and Tfh cell development.

IL-6 has multiple effects on immune and non-immune cells. IL-6 is implicated in the production of platelets, stem cell biology, bone formation, the acute phase response of hepatocytes, neural cell differentiation, cancer, keratinocyte biology and the growth of mesangial cells. It was first identified as a growth factor for B cells (Kinashi, T., et al. (1986). Cloning of complementary DNA encoding T-cell replacing factor and identity with B-cell growth factor II. Nature 324:70-73) and it is now known to promote the function of T helper cells that support B cells responses (Nurieva, R. I., et al. (2008). Generation of T follicular helper cells is mediated by interleukin-21 but independent of T helper 1, 2, or 17 cell lineages. Immunity 29:138-149). IL-6 is secreted by T cells and macrophages to stimulate an immune response to trauma, especially burns or other tissue damage leading to inflammation. In terms of host response to a foreign pathogen, IL-6 has been shown, in mice, to be required for resistance to multiple pathogens including the bacterium, Streptococcus pneumonia (van der Poll T, et al. (1997). “Interleukin-6 gene-deficient mice show impaired defense against pneumococcal pneumonia” J Infect Dis 176 (2): 439-44) as well as Toxoplasma gondii (Suzuki, Y., et al. (1997). Impaired resistance to the development of toxoplasmic encephalitis in interleukin-6-deficient mice. Infection Immunity 65:2339-2345). IL-6 can also be produced from muscle, and is elevated in response to muscle contraction (Febbraio M A, Pedersen B K (2005). “Contraction-induced myokine production and release: is skeletal muscle an endocrine organ?”. Exerc Sport Sci Rev 33 (3): 114-9). It is significantly elevated with exercise, and precedes the appearance of other cytokines in the circulation. IL-6 can also play a role as an anti-inflammatory cytokine through its inhibitory effects on TNF-alpha and IL-1, and activation of IL-1RA and IL-10. IL-6 is also an important mediator of fever because in muscle and fatty tissue IL-6 stimulates energy mobilization that can lead to increased body temperature. IL-6 can be secreted by macrophages in response to microbial molecules, and induce intracellular signaling cascades that give rise to inflammatory cytokine production.

IL-6 is also involved in, e.g., mediates, the pathology of a variety of diseases. For example, IL-6 is thought to promote the following: inflammatory bowel disease, Crohn's disease, multiple sclerosis, uveitis, psoriasis, arthritis, asthma, lupus, ulcerative colitis, Acute Disseminated Encephalomyelitis (ADEM), and transplant rejection, coronary heart disease, ischemic cerebrovascular disease, periodontal disease, angiogenesis, and cancer as well as many aspects of cellular growth and development including muscle and bone The present invention provides evidence that treating subjects with these conditions with p28 or a variant thereof, e.g., a variant with an increased half-life or altered binding properties, would reduce immune inflammation. Alternatively, antagonizing p28 (e.g., inhibiting or negatively modulating an activity of p28) can be used to augment a response mediated or promoted by IL-6. For example, during vaccination or immune mediated cancer therapy, a subject can be administered a p28 antagonist, e.g., a p28 inhibitor or a negative modulator of p28 activity, to increase the immune response.

T cells are critical mediators of disease, and much effort has focused on the development of strategies to specifically inhibit T cell responses. Many of the aforementioned IL-6-mediated diseases, e.g., inflammatory bowel disease, Crohn's disease, multiple sclerosis, uveitis, psoriasis, arthritis, asthma, lupus and transplant rejection are all conditions that involve T cells. For all of these conditions, there is a pressing need to develop new therapeutic approaches. In addition, T cells are required for the ability of B cells to produce certain classes of antibody, some of which may be auto-reactive (or auto-antibodies) that cause a variety of disease, many of which are not inflammatory in nature. The recognition that p28 antagonizes, or interferes with (e.g., inhibits) IL-6 mediated signaling, thereby limiting the ability of IL-6 to promote T and B cell responses, means that p28 represents a viable target to prevent these type of inflammatory responses. Alternatively, blockade of p28 could be used to augment T cell responses, for example during vaccination or immune mediated therapy for cancer. In addition, certain types of cancers, e.g., those associated with gp130 or IL-6 signaling, including but not limited to breast cancer, prostate cancer and gastric cancer, may also be susceptible to inhibitory signaling through this receptor.

gp130 (also known as gp130, IL6ST, IL6-beta or CD130) is the common signal transducer for several cytokines including leukemia inhibitory factor (LIF), ciliary neurotropic factor, oncostatin M, M-11, IL-12, IL-27, and IL-23 and cardiotrophin-1, and is almost ubiquitously expressed in most tissues. Multiple cytokines use gp130 combined with other receptor sub-units for signal transduction. Thus, any response mediated through a functional IL-6R or gp130, e.g., IL-12 and/or IL-23, can be modulated by activation or blockade of p28.

Various specific uses for p28 based on the novel strategies presented herein are described in more detail below.

### Commercially Useful Therapeutic Applications for p28

Several cytokine-specific antagonists are currently in development or commercially available for the treatment of a variety of diseases, including, e.g., ankylosing spondylitis, atherosclerosis, Crohn's disease, and chronic obstructive pulmonary disease (COPD). For example, Amgen, Merck/Schering-Plough, and Centrocor, among others, have developed antibodies that specifically antagonize, e.g., neutralize the biological effects, of the cytokine TNFα, and such antibodies are used to prevent TNFα-induced inflammation in the treatment of, e.g., Crohn's disease and rheumatoid arthritis. Similar therapeutic antibodies are being developed to neutralize and/or antagonize the activities of, e.g., IL-1, IL-12, p40 and IL-6 to treat and/or prevent a variety of diseases, including, e.g., rheumatoid arthritis, Crohn's disease, and systemic lupus erythematosus (SLE). However, therapeutic antibodies are expensive to produce. Furthermore, their imunogenicity limits long term use. In addition, the administration of therapeutic antibodies to subjects increases subjects' susceptibilities to opportunistic infections such as TB and toxoplasmosis (Bresnihan (2003) “Infection complications associated with the use of biologic agents.” Rheum Dis Clin North Am 29: 185-202). For these reasons, alternatives to antibody therapy are desirable.

The present application provides methods of using an endogenous protein, e.g., p28, to antagonize and/or neutralize the biological activities of IL-6 to treat and/or prevent a variety of diseases, including, but not limited to, e.g., cancer, autoimmune disease, transplant rejection, uveitis, Systemic lupus erythematosus (SLE), Autoimmune hepatitis, Bullous pemphigoid, Celiac disease, Guillain-Barré syndrome (GBS), Goodpasture's syndrome, Multiple sclerosis associated with the presence of autoantibodies to Myelin basic protein (MBP) and Myelin oligodendrocyte glycoprotein (MOG), Pemphigus Vulgaris, Primary biliary cirrhosis, Rheumatoid arthritis associated with rheumatoid factor, Scleroderma, or Wegener's granulomatosis. Using an endogenous signaling agent in treatment regimes can be more cost-effective and can preclude the undesirable side effects associated with the use of therapeutic antibodies.

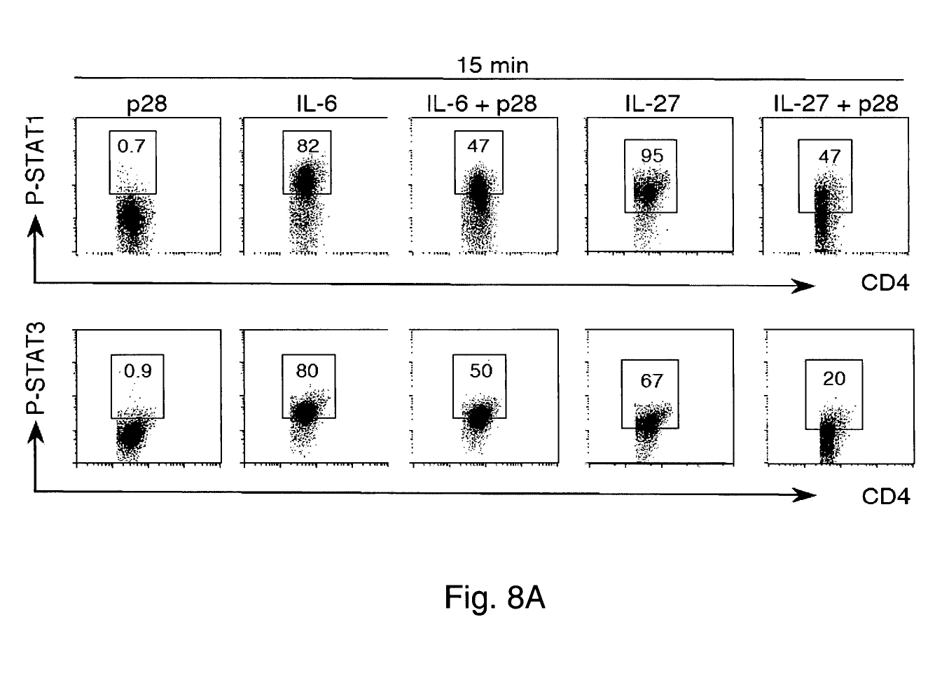

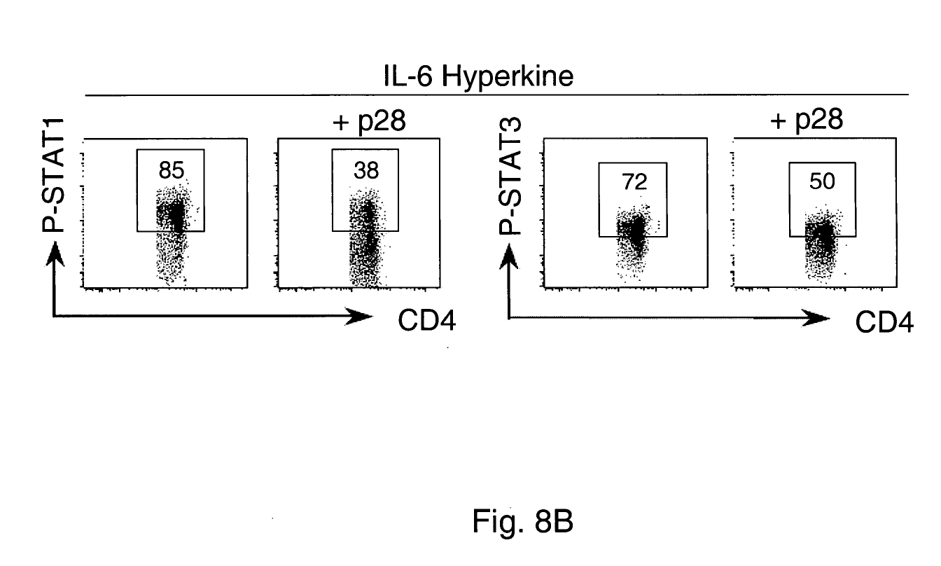

p28 was originally identified as a subunit of the heterodimeric type-1 cytokine IL-27 (which comprises p28 and EBI3). IL-27 plays a role in Th1 T cell differentiation and initiates its activity through a membrane bound receptor. (Further details regarding the IL27 receptor and IL27-mediated signaling are described in U.S. patent application Ser. No. 11/880,121, entitled, “WSX-1/p28 As a Target for Anti-Inflammatory Responses, by Hunter et al., filed Jul. 18, 2007). p28 can be secreted independently of EBI3 (Pflanz (2002) “IL-27, a heterodimeric cytokine composed of EBI3 and p28 protein, induces proliferation of naive CD4(+) T cells.” Immunity 16: 779-790) and has also been shown to inhibit IL-17 production by CD4+ T cells, thus influencing T-cell differentiation. As shown in the Example, p28 serves as an endogenous antagonist of gp130, the signal transducing subunit of the IL-6 receptor. Results described in the Example show that p28 inhibits IL-6 trans-signaling by binding to gp130, thus limiting the availability of this receptor subunit for binding to the IL-6 hyperkine, a fusion protein consisting of human IL-6 and the sIL-6Rα chain that only signals through gp130. Thus, p28 can be used to treat and/or prevent a variety of diseases that arise as the result of IL-6 mediated-signaling through gp130. In addition, p28 also block signals mediated by IL-27 (FIG. 8A) and it is possible that p28 can be used to antagonize signaling generally mediated by other cytokines that signal through gp130 such as LIF and IL-11. Moreover, it is also possible that p28 may block other related cytokine receptors

Deregulation of IL-6 production is implicated in the pathology of several disease processes, including, e.g., osteoporosis, psoriases, aging-related disease, etc. In one useful embodiment of the methods described herein, p28 can be administered directly to a subject, e.g., a human subject, that to suppress an IL-6 mediated inflammation response, thereby treating and/or preventing, e.g., atherosclerosis, cancer, autoimmune disease, lupus, multiple sclerosis (MS), rheumatoid arthritis (RA), asthma, uveitis, psoriasis, and transplant rejection, and any other disease associated with IL-6-mediated inflammation. Additionally, treatments for these diseases can also be improved by the co-administration of p28 with a currently available therapeutic. For example, the IL-1 receptor antagonist (IL-1RA) is used to treat arthritis, albeit with limited efficacy. IL-1RA treatment can be beneficially improved if it were administered in combination with an additional naturally occurring cytokine inhibitor, e.g., p28. In another embodiment of the methods of the invention, p28 can be administered in combination with other inflammation inhibitors, e.g., those that inhibit Th17 cells, to suppress many inflammatory conditions, including, but not limited to, e.g., SLE, autoimmune hepatitis, Bullous pemphigoid, Celiac disease, Guillain-Barré syndrome (GBS), Goodpasture's syndrome, multiple sclerosis associated with the presence of autoantibodies to myelin basic protein (MBP) and myelin oligodendrocyte glycoprotein (MOG), pemphigus vulgaris, primary biliary cirrhosis, rheumatoid arthritis associated with rheumatoid factor, scleroderma, or Wegener's granulomatosis.

IL-6 signaling is also associated with neuronal differentiation, hepatic regeneration (Tiberio et al. (2007) “Interleukin-6 sustains hepatic regeneration in cirrhotic rat.” Hepatogastroenterology 54: 878-883) and reduced insulin resistance (Senn (2002) “Interleukin-6 induces cellular insulin resistance in hepatocytes.” Diabetes 51: 3391-3399). Accordingly, in an alternative embodiment of the methods provided herein, an antagonist of p28, e.g., a modulator that decreases or inhibits p28 activity, can be administered to a subject to increase an IL-6 activity to treat, e.g., liver disease or diabetes. For example, IL-6 activity is associated with liver regeneration, thus blocking p28, which would increase IL-6 signaling, or not decrease IL-6 signaling, can be useful in treating liver disease. Similarly, as IL-6 is associated with reduced insulin resistance, blocking p28 can be useful in treating diabetes. An antagonist of p28 activity (e.g., a p28 inhibitor or a negative modulator of p28 activity) can also be beneficially administered to increase an IL-6 mediated immune response, as described below.

In our current model the different subunits of IL-27 and the different receptor chains of the IL-27 receptor have unique signaling functions and can affect distinct T cell functions. This concept leads to the design of molecules that affect T cell production of particular cytokines very specifically. Based exclusively on our work and data, we suggest that this approach can be used to target, for example, IL-27 signaling through gp130 or IL-6 signaling or indeed any other cytokine such as IL-11 and CNTF that signal through this complex. These represent valid drug targets for biotech and are important in many immune and non-immune conditions. See FIG. 1 for a schematic illustration of interactions in which IL-6 plays a role in the development of B cell responses. As discussed in greater detail and with additional examples herein, there are many additional approaches that can be formulated based on this information that allow us to rationally target discrete immune functions.

Preventing and Treating Inflammatory Conditions Using p28

Inflammation is a complex immunological response of vascular tissues to, e.g., pathogens, damaged cells, or irritants. Inflammation is a protective attempt by a host organism's immune system to remove such potentially deleterious stimuli and to initiate the healing process for, e.g., injured and/or infected tissue. Inflammation is regulated by a complex set of interactions between cytokines (see, e.g., Hanada et al. (2002) “Regulation of cytokine signaling and inflammation.” Cytokine Growth Factor Rev 13: 413-421; Yoshimura et al. (2003) “Negative regulation of cytokine signaling influences inflammation.” Curr Opin Immunol 15: 704-708; Odzemir et al. (2009) “T regulatory cells and their counterparts: masters of immune regulation.” Clin Exp Allergy 39: 626-39); and Elenkov et al. “Cytokine dysregulation, inflammation and well-being.” Neuroimmunomodulation 12: 255-269). IL-6 is produced at the site of inflammation and plays a key role in the acute phase inflammatory response, in which neutrophils migrate into damaged tissue. Additionally, IL-6 stimulates T- and B-cell proliferation and is involved in the maintenance of chronic inflammation. Although inflammation is an important component of the immune system's response to potentially harmful stimuli, the disregulation of the inflammatory response has also been implicated in, e.g., allergies, autoimmune disease, chronic infections, sepsis, atherosclerosis, rheumatoid arthritis, and cancer (Licastro et al. (2005) “Innate immunity and inflammation in ageing: a key for understanding age-related diseases.” Immunity and Ageing doi:10.1186/1742-4933-2-8; Coussens et al. (2003) “Inflammation and cancer.” Nature 422: 559; Rakoff-Nahoum (2006) “Why Cancer and Inflammation?” Yale J Biol Med 79: 123-130; and others). Moreover, using p28 transgenic mice that have B and T lymphocytes that over-express the p28 gene it was confirmed in in vitro experiments with recombinant murine p28 in the Example below that CD4⁺ T cells from the p28Tg mice produce less IL-17 and IL-10 in response to TGF-β and IL-6.

The present invention provides evidence that p28 can be advantageously used to prevent inflammation associated with IL-6 stimulated Th17 cells, which are associated with numerous inflammatory conditions (Egwuagu (2009) “STAT3 in CD4+ T helper cell differentiation and inflammatory diseases.” Cytokine 47: 149-156; Kim (2009) “Migration and function of Th17 cells.” Inflamm Alergy Drug Targ 8: 182-90). As described in further detail in the Example, this effect is likely the result of the antagonistic effect of p28 on IL-6 mediated Th17 differentiation. In one embodiment, the invention provides methods of suppressing an inflammatory immune response in a subject that include administering a combination of at least two inhibitors of Th17 differentiation. Typically, at least one of the two inhibitors of Th17 differentiation is p28. The second inhibitor can include, but is not limited to, an antagonist of IL-1, IL-21, TNF, or IL-23 or CTLA4-Ig. For example, in a preferred embodiment, the method comprises administering a combination of IL-1Ra and p28 to the subject. Details regarding the diagnosis of individuals who can benefit from the administration of p28 and the pharmaceutical administration of compositions comprising p28 are elaborated hereinbelow.

It will be understood by one of skill in the art that using a p28 to suppress or treat an inflammatory response includes using a variant p28, e.g., a p28 with an increased half-life. Further details regarding modified p28 (i.e., p28 variants) are discussed elsewhere herein.

Treating Cancer with p28

As described above, IL-6 is a multifunctional cytokine that plays a role in the regulation of both acute and chronic inflammatory responses. Elevated expression of IL-6 has been also detected in multiple types of tumors, e.g., breast, prostate, epithelial, and gastric tumors. See, e.g., Schaefer et al. (2007) “IL-6 involvement in epithelial cancers.” J Clin Invest 117: 3660-3663; Wang et al. (2009) “Inflammation and Cancer: IL-6 and STAT3 Complete the Link.” Cancer Cell 15: 79-80; Feurino et al. (2007) “IL-6 stimulates Th2 type cytokine secretion and upregulates VEGF and NRP-1 expression in pancreatic cancer cells.” Cancer Biol Ther 6: 1096-1100; Bollrath et al. (2009) “gp130-Mediated Stat3 Activation in Enterocytes Regulates Cell Survival and Cell-Cycle Progression during Colitis-Associated Tumorigenesis.” Cancer Cell 15: 91-102; and Ancrile et al. (2007) “Oncogenic Ras-induced secretion of IL6 is required for tumorigenesis.” Genes Dev 21: 1714-1719. IL-6 binds to a heterodimeric receptor that includes the ligand-binding IL-6α chain and the common cytokine receptor signal-transducing unit gp130. IL-6 receptor engagement leads to activation of the JAK family of tyrosine kinases, which then stimulate multiple pathways that include MAP kinases, PI3 kinases, STATs, and other signaling proteins involved in cell proliferation (Hong et al. (2007) “Interleukin-6 and its receptor in cancer: implications for translational therapeutics.” Cancer doi:10.1002/cncr.22999). IL-6-mediated signaling has also been implicated in tumorigenesis (Hodge et al (2005) “The role of IL-6 and STAT3 in inflammation and cancer.” Eur Journal Cancer 41: 2502-2512). As p28 is an antagonist of IL-6 mediated signaling, therapeutic compositions that include p28 can be useful in treating and/or preventing cancers.

For example, individuals with predispositions for developing cancers associated with hyperactivation of the gp130 signaling pathway (e.g., mediated through IL-6 or other cytokines such as IL-11) or individuals diagnosed with cancer who exhibit gp130 hyperactivation represent target populations that can be beneficially treated with p28 or a p28 variant, e.g., alone or in combination with other antagonists of cytokine mediated signaling or current chemotherapeutic regimes. Other such antagonists can include, e.g., Avastin or anti-HER2/neu, or anti-angiogenic treatments, e.g., anti-VEGF. In certain embodiments, methods of treating an IL-6 or gp130-mediated cancer typically comprise identifying a subject who has or is predisposed to develop a gp130-associated cancer; and administering p28 to the subject, e.g., p28 without EBI3. An additional gp130 antagonist or cytokine antagonist is also optionally administered in combination with p28. Any current cancer treatment, e.g., herceptin treatment, can be combined with the administration of p28. Details regarding the diagnosis of individuals who can benefit from the administration of p28 and the pharmaceutical administration of compositions comprising p28 are elaborated hereinbelow.

Using p28 to Treat and/or Prevent Autoimmune Diseases

IL-6 is also implicated in the pathology of several autoimmune diseases, including, e.g., rheumatoid arthritis (RA), inflammatory bowel disease, systemic-onset juvenile chronic arthritis (JCA), systemic lupus erythematosus (SLE), Crohn's disease, multiple sclerosis, psoriasis, and others (Mudter et al. (2007) “11-6 signaling in inflammatory bowel disease: pathophysiological role and clinical relevance.” Inflamm Bowel Dis 13: 1016-23; Bongioanni et al. (2000) “Increased T-lymphocyte interleukin-6 binding in patients with multiple sclerosis.” Eur J Neurol. 7: 291-297). For example, elevated levels of IL-6 in serum, urine and renal glomeruli are detected in patients with active SLE and in murine models of SLE (Liang et al. (2006) “Anti-interleukin-6 monoclonal antibody inhibits autoimmune responses in a murine model of systemic lupus erythematosus.” Immunology 119: 296-305). IL-6 is critically involved in experimentally induced autoimmune disease, such as antigen-induced arthritis (AIA), and experimental allergic encephalomyelitis.

The cytokine IL-6 is a B cell growth factor and plays an important role in the development of the Tfh cell-dependent B cell activation. Additionally, IL-6 and also acts as a growth factor for B cells. B cells are thought to contribute to autoimmunity through several mechanisms: production of auto-antibodies; antigen presentation and co-stimulation during initiation of immune responses; regulation of secondary lymphoid tissue organization and neogenesis; and release of inflammatory and immunomodulatory cytokines. In conditions where B cells and their production of auto-antibodies leads to the development of disease, antagonizing IL-6 is a useful way to either prevent the development of disease or ameliorate this condition. Data in the Example show that p28 transgenic mice are unable to form GC reactions, which are necessary for B cell class switching and affinity maturation to occur in response to immunization with the T cell dependent antigen NP-CGG. Thus, p28 and/or a modified p28 variant, e.g., comprising one or more conservative amino acid substitutions, can be advantageously administered to a subject, e.g., a human patient, to suppress of B cell activity. SLE, autoimmune hepatitis, bullous pemphigoid, celiac disease, Guillain-Barré syndrome (GBS), Goodpasture's syndrome, multiple sclerosis associated with the presence of autoantibodies to MBP and MOG, pemphigus vulgaris, primary biliary cirrhosis, rheumatoid arthritis associated with rheumatoid factor, scleroderma, or Wegener's granulomatosis can be treated by the therapeutic administration of p28. Data presented herein indicate that p28 is a potent antagonist of IL-6 mediated signaling and is useful alone or in combination with other antagonists of T cell/B cell interactions (for example ICOS-ICOS-L; IL-21, CD40-CD40L) to limit Tfh activation and growth as well as limiting B cell growth and auto-antibody production.

For example, the present invention provides a method of treating autoimmune diseases using p28 (e.g., a modified p28) or by combining p28 (or a modified p28, e.g., a p28 variant comprising, e.g., at least one conservative amino acid substitution) with other antagonists of the Th17 pathway. This includes, but is not limited to antagonists of IL-1, IL-21, TNF and IL-23 cytokine/cytokine receptor interactions. These types of combinations provide a more potent strategy to limit inflammation mediated by these IL-6-driven Th17 cells than previously available.

In some embodiments, an additional antagonist of T and B cell interactions is administered with the p28. The additional antagonist blocks an interaction of ICOS-ICOS-L, IL-21, or CD40-CD40L.

Limiting Transplant Rejection

One of the factors that currently limits the success of organ transplantation is rejection, e.g., wherein the organ recipient's immune system attacks the transplanted organ just as it would attempt to destroy foreign infecting organisms. While immune-mediated allograft or xenograft rejection can be currently controlled with cocktails of immunosuppressive drugs, the drugs, which can cause numerous undesirable side effects, must be administered for the life of the transplantation (and/or the patient). The induction and maintenance of immune tolerance to transplanted tissues constitute an active process involving multiple mechanisms that work cooperatively to prevent graft rejection. These mechanisms are similar to inherent tolerance toward self-antigens (described above) and entail T-cell mediated immunoregulation that promotes specific unresponsiveness to antigens present on the transplanted organ. Many transplant models have shown the importance of a subset of T lymphocytes, termed Treg in limiting alloreactive immunity (Walsh et al. (2004) “Tregs and transplantation tolerance.” J Clin Invest 114: 1398-1403; Benard et al. (2006) “Regulatory T cells control autoimmunity following syngeneic bone marrow transplantation.” Eur J Immunol 36: 2324-2335; Wood (2003) “Regulatory T cells in transplantation tolerance.” Nat Rev Immunol 3: 199-210). However, IL-6 has been found to promote differentiation of naïve T lymphocytes into pro-inflammatory IL-17 cytokine-producing T helper 17 (Th-17) cells, which promote autoimmunity and inflammation (Korn et al. (2008) “IL-6 controls Th17 immunity in vivo by inhibiting the conversion of conventional T cells into Foxp3+ regulatory T cells.” Proc Natl Acad Sci USA 105: 18460-5; Pasare et al. (2003) “Toll pathway-dependent blockade of CD4⁺CD25⁺ T cell-mediated suppression by dendritic cells.” Science 299: 1033-1036; Afzali et al. (2007) “The role of T helper 17 (Th17) and regulatory T cells (Treg) in human organ transplantation and autoimmune disease.” Clin Exp Immunol 148: 32-46).

Methods provided by the invention can be useful in inhibiting the IL-6-driven induction of pro-inflammatory Th-17 cells and promoting anti-inflammatory Treg differentiation to suppress transplant rejection. p28 can antagonize, e.g., inhibit, IL-6 signaling through gp130, thereby stimulating the differentiation of Treg cells that act to suppress activation of the immune system. For example, the administration of p28 (or a p28 variant), e.g., alone or in combination with other cytokines or immunosuppressants, to the recipient of an organ transplant can beneficially block IL-6 mediated signaling. For example, an isolated p28, e.g., not in complex with EBI3, can be administered to prevent or treat graft rejection.

Enhancing IL-6 Mediated Immune Responses

IL-6 signaling is also associated with neuronal differentiation, hepatic regeneration (Tiberio et al. (2007) “Interleukin-6 sustains hepatic regeneration in cirrhotic rat.” Hepatogastroenterology 54: 878-883) and reduced insulin resistance (Senn (2002) “Interleukin-6 induces cellular insulin resistance in hepatocytes.” Diabetes 51: 3391-3399). Accordingly, in an alternative embodiment of the methods provided herein, a modulator that decreases p28 activity can be administered to a subject to increase an IL-6 activity to treat, e.g., liver disease or diabetes. In another aspect, the present invention provides a method of enhancing an IL-6 mediated immune response in a subject. The method comprises administering an antagonist of p28 to the subject (e.g., administering a p28 inhibitor or negative modulator of p28 activity) which allows IL-6 mediated signaling, e.g., through gp130, to occur more efficiently. As p28 inhibits IL-6 from activating T cells, an p28 antagonist, a p28 inhibitor, or negative modulator of p28 activity can be used to activate the immune system. For example, a subject, e.g., a human patient, who is the recipient of a vaccination for infectious disease or immune mediated cancer therapy would benefit from an activated immune response. Therefore, the present invention provides a method of enhancing an immune response by administering an antagonist of p28. Alternatively, blocking p28 can be useful to promote Tfh development and to allow IL-6 to limit Treg differentiation

### p28 Therapeutic Treatments

One aspect of the invention provides methods of treating an immune mediated disease, an inflammatory condition, cancer, or any other disease mediated by IL-6 or gp130 signaling in a mammalian subject, e.g., a human subject, by administering p28 or a p28 variant, e.g., a variant with increased half life or a variant comprising one or more conservative amino acid substitutions. The condition to be treated can be essentially any condition affected by IL-6 mediated signaling. The condition is optionally T cell-mediated; for example, the condition can be mediated by T_{H}1 cells, T_{H}2 cells, T17 cells, T_{H}-17 cells, CD4⁺ T cells, CD8⁺ T cells, gamma/delta T cells, natural killer T cells, and/or regulatory T cells. Exemplary inflammatory conditions to be treated include, but are not limited to, autoimmune diseases such as systemic lupus erythematosus (SLE), autoimmune hepatitis, bullous pemphigoid, celiac disease, Guillain-Barré syndrome (GBS), Goodpasture's syndrome, multiple sclerosis associated with the presence of autoantibodies to Myelin basic protein (MBP) and myelin oligodendrocyte glycoprotein (MOG), pemphigus vulgaris, primary biliary cirrhosis, rheumatoid arthritis associated with rheumatoid factor, scleroderma, or Wegener's granulomatosis.

In one class of embodiments, the methods include administering to the subject an isolated or recombinant p28 moiety or, e.g., a p28 variant moiety. In embodiments in which a combination of recombinant or isolated polypeptides are administered (e.g., a p28 polypeptide and an additional T or B cell antagonist), the polypeptides can but need not form a complex, and the polypeptides can be co-administered or separately administered. It will be understood by one of skill in the art that using a p28 to suppress or treat an inflammatory response includes using a modified p28, e.g., a p28 variant with an increased half-life. Further details regarding p28 variants are discussed elsewhere herein.

In another class of embodiments, the methods include administering to the subject a moiety that specifically antagonizes p28 activity, e.g., by binding to or modulating an activity of p28, by modulating formation of or expression of p28 in a cell. The moiety can be, for example, an antibody, an antagonist, an agonist, a nucleic acid, a small organic molecule an activity modulator, or the like.

In either class of embodiments, the methods optionally include diagnosing the patient with the inflammatory condition and/or disease state, e.g., those listed above, prior to said administering. In the present invention diagnosing optionally includes determining whether IL-6 mediated signaling is involved with the inflammatory condition to be treated or prevented. Such assays are discussed in, e.g., Thibault (1997)“Antibodies to rat soluble IL-6 receptor stimulate B9 hybridoma cell proliferation.” FEBS Lett 408: 182-186; and others.

In addition to diagnosing a patient with an inflammatory condition, a cancer, an autoimmune disease, etc., the level of p28 can be used to diagnose disease state, extent of disease or to determine what type of immune mediation is most appropriate. For example, the ratio of different cytokines can be predictive of cause of disease (Tuma, R., N. et al. 2006. The serum IL-12:IL-6 ratio reliably distinguishes infectious from non-infectious causes of fever during autologous stem cell transplantation. Cytotherapy 8:327-334). Accordingly, the level of, e.g., serum p28 can provide a useful criterion for determining the pathology of a disease. For example, if p28 levels are unusually high, or, alternatively, unusually low, this information can inform the determination of a diagnosis, a prognosis, or a therapeutic regimen. For example, a Crohn's disease patient who exhibits high levels of p28 might not benefit from the administration of a drug that further suppresses IL-6 signaling, but, rather, may benefit from administration of a drug that suppresses the activity of another inflammatory cytokine, e.g., TNF. Alternatively, a Crohn's patient that exhibits low levels of p28 could be supplemented with p28, e.g., alone or in combination with another IL-6 signaling inhibitor or inflammatory cytokine inhibitor. The level of p28 becomes an indicator of which is the better treatment option.

In another embodiment, high serum levels of p28 are associated with or indicative of different disease states. For example, a disease such as SLE may be diagnosed based on a combination of criteria such as the presence of anti-nuclear antibodies as well as altered levels of p28. Also, the relative ratio of p28 to other cytokines (IL-12/IL-6) can be used to indicate disease state or treatment options.

Once diagnosed as being in need of immune suppression or immune mediation, a therapeutically effective amount of the p28 moiety can be administered to the subject. Optionally, the subject is monitored for response to the treatment. In one class of embodiments, after initiation of treatment the subject displays decreased inflammation, for example, reduced numbers of inflammatory cells, a reduction in the number of IL17⁺ T cells in circulation or at the site of inflammation, and/or reduced production of auto-reactive antibodies.

It will be evident that relevant complexes can optionally be formed in vivo. For example, in embodiments in which a polypeptide is administered, the polypeptide can form an active complex with endogenous protein(s). As one example, when a soluble p28 polypeptide is administered to the subject, it can form a complex with endogenous EB13 and/or IL-27, leading to therapeutic results. A polypeptide to be administered is optionally a modified polypeptide variant having a higher affinity for the receptor components, e.g., than wild-type protein (e.g., a p28 variant having a higher affinity for WSX-1 or the WSX-1/gp130 receptor complex than does a corresponding naturally occurring p28 from which the variant is derived). Optionally, a modified p28, i.e., a p28 variant will exhibit an increased half-life relative to a the wild type p28 from which it was derived.

In one aspect, the methods include administering to the subject a therapeutically effective amount of a combination of, e.g., a p28 polypeptide, complex, or variant thereof, e.g., a modified p28 polypeptide, and at least a second compound. The second compound is typically one that is used to treat the inflammatory condition, for example, a standard of care or experimental treatment. Exemplary second compounds include, but are not limited to, immune modulators that affect IL-23, IL-12, IL-6 or TGF (e.g., antibodies specific to IL-12 p40, p35 or IL-23 p19); antibodies or reagents that antagonize the functions of IL-1 (e.g., anakinra (Kineret®), soluble IL-1 receptor) and TNF (e.g., anti-TNF antibodies, etanercept, infliximab, and leflunomide); a cytotoxic agent; an immunosuppressive agent (e.g., cyclophosphamide); a B-cell surface marker antagonist; an antibody to a B-cell surface marker; a CD20 antibody, e.g., Rituximab, see US 20060051345); a CD5, CD28, or CD40 antibody or blocking agent; a corticosteroid (e.g., prednisone), CTLA4-Ig, an alpha4-integrin antibody or antagonist such as natalizumab (Tysabri®), mycophenolate mofetil, a statin, an LFA-1 or CD-11a antibody or blocking agent (see U.S. patent application publication 20050281817 by Jardieu et al. entitled “Method for treating multiple sclerosis”), an interleukin-12 antibody, a beta interferon (e.g., an interferon β-1a such as Avonex® or Rebif®, or an interferon β-1b such as Betaseron®), glatiramer acetate (Copaxone®), a CD52 antibody such as alemtuzuman (CamPath®), an interleukin receptor antibody such as daclizumab (Zenapax®, an antibody to the interleukin-2 receptor alpha subunit), etc. In one class of embodiments, the second compound is an additional T or B cell antagonist, e.g., an antagonist that blocks the interaction of ICOS-ICOS-L, IL-21, or CD40-CD40L, or an inhibitor of Th17 differentiation, e.g., IL-1RA, an Il-21, IL-23, TNF, or CTLA4-Ig. In other embodiments, p28 is combined with a monoclonal antibody, e.g., herceptin, for improved anti-cancer treatment. Preferably, the compositions and methods herein do not involve administering p28 in a complex with, e.g., EB13, gp130, or WSX-1.

In one embodiment, the subject has never been previously treated with drug(s) to treat the inflammatory condition and/or has never been previously treated with a moiety of the invention. In another embodiment, the subject has been previously treated with drug(s) to treat the inflammatory condition and/or has been previously treated with such moiety.

Typically, the subject is eligible for treatment for the immune-mediated condition, i.e., an eligible subject. For the purposes herein, such eligible subject is one who is experiencing, has experienced, or is likely to experience, one or more signs, symptoms or other indicators of the inflammatory condition; has been diagnosed with the inflammatory condition, whether, for example, newly diagnosed, previously diagnosed with a new relapse or exacerbation, previously diagnosed and in remission, etc; and/or is at risk for developing an IL-6 related disease, e.g., cancer, transplant rejection, an inflammatory condition, an autoimmune disease, Systemic lupus erythematosus (SLE), Autoimmune hepatitis, Bullous pemphigoid, Celiac disease, Guillain-Barré syndrome (GBS), Goodpasture's syndrome, Multiple sclerosis associated with the presence of autoantibodies to Myelin basic protein (MBP) and Myelin oligodendrocyte glycoprotein (MOG), Pemphigus Vulgaris, Primary biliary cirrhosis, Rheumatoid arthritis associated with rheumatoid factor, Scleroderma, or Wegener's granulomatosis. Optionally, administration of, e.g., a p28 polypeptide, complex, or variant thereof, e.g., a p28 polypeptide variant, can be used to treat a subject exhibiting symptoms of more than one IL-6 signaling mediated disease.

Administration

As will be understood by those of ordinary skill in the art, the appropriate doses of the appropriate moieties (e.g., p28 polypeptides, complexes, or variants thereof, e.g., modified p28 polypeptides comprising one or more conservative amino acid substitutions) will be generally around those already employed in clinical therapies wherein similar moieties are administered alone or in combination with other therapeutics. Variation in dosage will likely occur depending on the condition being treated. The physician administering treatment will be able to determine the appropriate dose for the individual subject. Preparation and dosing schedules for commercially available second compounds administered in combination with the moieties may be used according to manufacturers' instructions or determined empirically by the skilled practitioner.

For the prevention or treatment of disease, the appropriate dosage of the moiety (e.g., a p28 polypeptide, complex, or variant thereof, e.g., a modified p28 polypeptide) and any second compound administered in combination with the moiety will depend on the type of disease to be treated, as defined above, the severity and course of the disease, whether the moiety or combination is administered for preventive or therapeutic purposes, previous therapy, the patient's clinical history and response to the antibody or combination, and the discretion of the attending physician. The moiety or combination is suitably administered to the patient at one time or more typically over a series of treatments. The complexes administered herein do not typically include EBI3, gp130, or WSX-1.

Depending on the type and severity of the disease, about 1 μg/kg to 50 mg/kg (e.g. 0.1-20 mg/kg) of the moiety is an initial candidate dosage for administration to the patient, whether, for example, by one or more separate administrations, or by continuous infusion. A typical daily dosage might range from about 1 μg/kg to about 100 mg/kg or more, depending on the factors mentioned above. For repeated administrations over several days or longer, depending on the condition, the treatment is sustained until a desired suppression of disease symptoms occurs. However, other dosage regimens may be useful. Typically, the clinician will administer a moiety of the invention (alone or in combination with a second compound) until a dosage(s) is reached that provides the required biological effect. The progress of the therapy of the invention is easily monitored by conventional techniques and assays.

The moiety can be administered by any suitable means, including parenteral, topical, subcutaneous, intraperitoneal, intrapulmonary, intranasal, and/or intralesional administration. Parenteral infusions include intramuscular, intravenous, intradermal, intraarterial, intraperitoneal, or subcutaneous administration. Intrathecal administration is also contemplated (see, e.g., U.S. patent application publication 2002/0009444 by Grillo-Lopez). In addition, the moiety may suitably be administered by pulse infusion, e.g., with declining doses of the moiety. Optionally, the dosing is given intravenously or subcutaneously, and optionally by intravenous infusion(s). Each exposure may be provided using the same or a different administration means. In one embodiment, each exposure is by intravenous administration.

As noted, the moiety can be administered alone or in combination with at least a second compound. These second compounds are generally used in the same dosages and with administration routes as described above or known to those of skill in the art, or about from 1 to 99% of the heretofore-employed dosages. If such second compounds are used, optionally they are used in lower amounts than if the moiety were not present, so as to eliminate or reduce side effects caused thereby. Such second compounds to not typically include gp130, WSX-1, and/or EBI3.

The administration of the moiety of the invention and any second compound can be done simultaneously, e.g., as a single composition or as two or more distinct compositions using the same or different administration routes. Alternatively, or additionally, the administration can be done sequentially, in any order. In certain embodiments, intervals ranging from minutes to days, to weeks to months, can be present between the administrations of the two or more compositions. For example, the moiety may be administered first, followed by the second compound of the invention. However, simultaneous administration or administration of the second compound of the invention first is also contemplated. For example, a second compound is optionally an additional T or B cell antagonist, e.g., an antagonist that blocks the interaction of ICOS-ICOS-L, IL-21, or CD40-CD40L, or an inhibitor of Th17 differentiation, e.g., IL-1RA, an Il-21, IL-23, TNF, or CTLA4-Ig.

A third, fourth, etc. compound is optionally administered in combination with the moiety and the second compound. Similarly, treatment for symptoms secondary or related to the inflammatory condition (e.g., spasticity, incontinence, pain, fatigue, etc.) can be administered to the subject, e.g., during treatment with the moiety or combination.

Pharmaceutical Formulations

Therapeutic formulations of the moieties of the invention (e.g., p28 polypeptides, modified p28 polypeptide variants, complexes, antibodies, that increase or, alternately, decrease p28 levels or activity, etc.) used in accordance with the present invention are prepared for storage by mixing a moiety, having the desired degree of purity with optional pharmaceutically acceptable carriers, excipients, or stabilizers (Remington's Pharmaceutical Sciences 16th edition, Osol, A. Ed. (1980)), in the form of lyophilized formulations or aqueous solutions. Acceptable carriers, excipients, or stabilizers are nontoxic to recipients at the dosages and concentrations employed, and include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low-molecular-weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as Tween®, Pluronics®, or PEG.

Lyophilized formulations adapted for subcutaneous administration are described, for example, in U.S. Pat. No. 6,267,958 (Andya et al.). Such lyophilized formulations may be reconstituted with a suitable diluent to a high protein concentration and the reconstituted formulation may be administered subcutaneously to the mammal to be treated herein. Crystallized forms of the moiety are also contemplated. See, for example, U.S. 2002/0136719A1 (Shenoy et al.).

The formulation herein may also contain at least a second compound as necessary for the particular indication being treated, preferably those with complementary activities that do not adversely affect each other. For example, it may be desirable to further provide transforming growth factor beta (TGF-β), a cytotoxic agent (e.g. methotrexate, cyclophosphamide, or azathioprine), chemotherapeutic agent, immunosuppressive agent, cytokine, cytokine antagonist or antibody, growth factor, hormone, integrin, integrin antagonist or antibody (e.g., an LFA-1 antibody, or an alpha 4 integrin antibody such as natalizumab), interferon class drug such as IFN-beta-1a or IFN-beta-1b, an oligopeptide such as glatiramer acetate, intravenous immunoglobulin (gamma globulin), lymphocyte-depleting drug (e.g., mitoxantrone, cyclophosphamide, CamPath® antibodies, or cladribine), non-lymphocyte-depleting immunosuppressive drug (e.g., MMF or cyclosporine), cholesterol-lowering drug of the “statin” class, estradiol, drug that treats symptoms secondary or related to lupus or MS (e.g., spasticity, incontinence, pain, fatigue), a TNF inhibitor, disease-modifying anti-rheumatic drug, nonsteroidal antiinflammatory drug, corticosteroid (e.g., methylprednisolone, prednisone, dexamethasone, or glucorticoid), levothyroxine, cyclosporin A, somatastatin analogue, anti-metabolite, a T- or B-cell surface antagonist/antibody, etc., or others as noted above in the formulation. The type and effective amounts of such other agents depend, for example, on the amount of moiety present in the formulation, the type of inflammatory condition being treated, and clinical parameters of the subjects.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug-delivery systems (for example, liposomes, albumin microspheres, microemulsions, nano-particles and nanocapsules) or in macroemulsions. Such techniques are disclosed, e.g., in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and y ethyl-L-glutamate, non-degradable ethylene-vinyl acetate, degradable lactic acid-glycolic acid copolymers such as the Lupron Depot® (injectable microspheres composed of lactic acid-glycolic acid copolymer and leuprolide acetate), and poly-D-(−)-3-hydroxybutyric acid.

The formulations to be used for in vivo administration must be sterile. This is readily accomplished by filtration through sterile filtration membranes.

### Screening for Modulators of p28

Compounds that negatively modulate (e.g., antagonize or inhibit) the activity of, p28, EBI3, and/or gp130 can be useful, for example, enhancing inflammation or otherwise enhancing an IL-6 mediated immune response. Accordingly, it can be useful to identify a compound that binds to or modulates an activity of a modified p28 variant, e.g., a modified p28 polypeptide comprising one or more conservative amino acid substitutions. In the methods, a biological or biochemical sample comprising the polypeptide or complex is contacted with a test compound. Binding of the test compound to the polypeptide or complex or modulation of the activity of the polypeptide or complex by the test compound is detected, thereby identifying the compound that binds to or modulates the activity of the polypeptide or complex, e.g., a p28 or a variant thereof.

In one class of embodiments, the compound potentiates inhibition of IL-6 signaling by p28 or a variant thereof, potentiates antagonist activity against IL-6, or alters T cell proliferation, survival, or expression of IL-6 relative to a corresponding T cell not treated with the compound. The compound optionally decreases expression of p28, decreases interaction of p28 with WSX-1 or the IL-27 receptor, or the like. Optionally, the compound potentiates inhibition of a p28-mediated T cell response, potentiates antagonist activity against IL-2 or IL-17, or alters expression of IL-2, IFN-gamma, TNF-alpha, IL-6, IL-4, IL-13, IL-17, IL-25, IL-10, IL-5, or CD25 relative to a corresponding T cell not treated with the compound. The compound optionally binds to p28, a modified p28 variant, the IL-27 receptor, blocks interaction between WSX-1 and gp130, potentiates interaction between WSX-1 and gp130, potentiates interaction of p28 with WSX-1 or the IL-27 receptor, or the like. Exemplary compounds include antibodies (e.g., antibodies against WSX-1, p28, EBI3, and/or gp130 polypeptides), agonists, antagonists, and activity modulators, for example, small organic molecules nucleic acids, proteins, ligands, and the like.

The biological or biochemical sample can include isolated or recombinant polypeptides or complexes, cells (e.g., T-cells), tissue samples, and/or the like. T cell responses such as proliferation, survival, and marker expression can be assayed by techniques known in the art.

Such modulators can be administered to an eligible subject, e.g., to increase or decrease an immune response or an inflammation response, as needed.

### p28 Compositions

Compositions comprising p28 decrease inflammation when administered to a subject, e.g., a human or animal exhibiting inflammation prior to such administration. Results described in the Example indicate that the p28 produced by the transgenic cells was able to efficiently antagonize the activity of IL-6 and IL-27 in vitro similar to what was observed for recombinant p28. The compositions alter the ability of IL-6 to promote a gp-130-mediated T cell response and/or inflammation response, in cells to which the composition is applied relative to cells not exposed to the composition. The compositions optionally include a pharmaceutically acceptable excipient, for example, in embodiments in which the composition is to be administered to a subject, e.g., a human subject. In one embodiment, a composition comprising a p28 polypeptide suppresses T follicular helper cells, whose proliferation are stimulated by IL-6-mediated signaling.

Compositions that include p28 can also optionally include one or more cell, for example, one or more T cell, B cell, mast cell, neutrophil, macrophage, dendritic cell, or other cell expressing gp130 alone (e.g., endothelial cell) or in combination with any other functional cytokine receptor sub-unit. The complex can affect a function or activity of the cell. In one embodiment, the composition includes a T-cell, and the composition alters a function or activity of the T-cell, relative to a corresponding T-cell not treated with the composition. For example, the T-cell can display altered expression, altered proliferation, or altered survival. Expression of various cytokines can be detected by any of a variety of techniques well known in the art, e.g., for detecting mRNA and/or protein levels. IL-6 signaling is typically downregulated by the compositions of the invention.

Suitable p28 polypeptides include, for example, the entire p28 sequence or any active fragment thereof and optionally portions of the EB13 subunit or a portion (a subsequence) thereof. The p28 polypeptides are optionally part of a fusion protein, e.g., one of those described herein or a fusion with a Fc region, e.g., an IgG Fc domain. See, for example, U.S. patent application publication 20040185049 by Hunter and Villarino entitled “Methods for modulating an inflammatory response” and Wirtz et al. “Protection from lethal septic peritonitis by neutralizing the biological function of interleukin 27” J. Exp. Med. 10.1084/jem.20060471. p28 polypeptides are readily constructed, and some are commercially available. For example, human and mouse IL-27 are available from R&D Systems (on the web at www (dot) rndsystems (dot) corn). Similarly, suitable p28 and EBI3 polypeptides include p28 or a subsequence thereof. The components of the complex are optionally noncovalently associated in the complex, or are optionally covalently connected by a chemical crosslinker or the like in the complex. Complexes of the invention do not include, e.g., EBI3, gp30, or WSX-1.

Fusion proteins are another feature of the invention. Accordingly, certain p28 compositions can optionally include a recombinant or isolated p28 fusion protein. The fusion protein includes a p28 polypeptide, which can be, e.g., at the N-terminus of the fusion protein, at the C-terminus of the fusion protein, or internal to the fusion protein.

Optionally, the fusion protein comprises one or more domains that recognize a cell-specific marker, for example, one or more antibody domains (e.g., V_{H} and V_{L} domains) that recognize the marker. The cell-specific marker can be essentially any cell-specific marker, for example, a marker for a lymphocyte population, a T cell, a cell of the innate immune response such as a neutrophil, dendritic cell, or mast cell, or a cancer cell. A variety of such markers for various cell types are known in the art, and more can be determined by techniques well known in the art. In one class of embodiments, the cell-specific marker is selected from CD4, CD8, CD11c, CD11b, and NK1.1.

Optionally, a fusion protein comprises one or more polypeptide domains derived from p28. A heterologous polypeptide domain can be joined to p28 through a linker. Many suitable linkers are known in the art (e.g., linkers including 4-6 Gly and/or Ala residues), and additional linkers are readily designed (see, e.g., Crasto and Feng (2000) “LINKER: A program to generate linker sequences for fusion proteins” Protein Engineering 13:309-312). However, the naturally occurring subunit p28 alone is a preferred embodiment.

A fusion protein of the invention can be monomeric, dimeric (e.g., homodimeric or heterodimeric), or multimeric. The fusion protein is preferably soluble. Optionally, the fusion protein forms a complex with EBI3 or IL-27.

Optionally, a p28 composition can include a recombinant or isolated p28 fusion protein. The fusion protein includes a p28 polypeptide, which can be, e.g., at the N-terminus of the fusion protein, at the C-terminus of the fusion protein, or internal to the fusion protein. The p28 polypeptide can be derived from a naturally occurring p28 (e.g., human p28) or a variant thereof.

D28 Polypeptide Variants

In other p28 compositions, suitable p28 polypeptides include modified p28 polypeptide variants. For example, amino acid sequence modification(s) of a p28 protein or peptide fragment are contemplated. For example, it may be desirable to improve the half-life and/or other biological properties of the p28 polypeptide. Amino acid sequence variants of a p28 polypeptide, e.g., modified p28 polypeptides, are prepared by introducing appropriate nucleotide changes into the p28 polypeptide-encoding nucleic acid, or by peptide synthesis. Such modifications include, for example, deletions from, and/or insertions into and/or substitutions of, residues within the wild type p28 amino acid sequence. Any combination of deletion, insertion, and substitution is made to arrive at the final construct, provided that the final construct possesses the desired characteristics, e.g., wild-type p28 binding activity with respect to EBI3, WSX-1, and/or gp130. The amino acid changes also may alter post-translational processing of the p28 polypeptide.

A useful method for identification of certain residues or regions of the p28 polypeptide that are preferred locations for mutagenesis is called “alanine-scanning mutagenesis” as described by Cunningham and Wells Science, 244:1081-1085 (1989). Here, a residue or group of target residues are identified (e.g., charged residues such as Arg, Asp, His, Lys, and Glu) and replaced by a neutral or negatively charged amino acid (most preferably alanine or polyalanine) to affect an activity of the p28 polypeptide. Those amino acid locations demonstrating functional sensitivity to the substitutions then are refined by introducing further or other variants at, or for, the sites of substitution. Thus, while the site for introducing an amino acid sequence variation is predetermined, the nature of the mutation per se need not be predetermined. For example, to analyze the performance of a mutation at a given site, ala scanning or random mutagenesis is conducted at the target codon or region and the expressed p28 variants (e.g., modified p28 polypeptides) are screened for a desired activity, as described in the Example below.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an modified p28 polypeptide variant with an N-terminal methionyl. Other insertional variants of the modified p28 polypeptide include the fusion to the N- or C-terminus of an enzyme, or a polypeptide that increases the serum half-life of p28.

Another type of variant is an amino acid substitution variant. These variants have at least one amino acid residue in the modified p28 polypeptide replaced by a different residue. Such substitutions can be conservative or nonconservative, as long as they preserve WT activity, as described in the Example.

Nucleic acid molecules encoding amino acid sequence variants of modified p28 polypeptides are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of an earlier prepared variant or a non-variant version of the p28 polypeptide.

### Recombinant Cells and Non-Human Animals

Transgenic Laboratory Animals

Transgenic (non-human) laboratory animals such as mice and other rodents are useful tools for studying gene function and for testing p28 modulators and p28 variants. Human (or other selected) cytokine genes and/or cytokine receptor genes can also be introduced in place of endogenous genes of a laboratory animal, making it possible to study function of the human (or other) cytokine in the easily manipulated and studied laboratory animal. Although similar genetic manipulations can be performed in tissue culture, the interaction of IL-6, IL-27, etc. with recombinant, e.g., a human p28 or variants thereof, in the context of an intact organism, provides a more complete and physiologically relevant picture of the effects of p28 modulation, overexpression, etc., than can be achieved in simple cell-based screening assays. Accordingly, one feature of the invention is the creation of transgenic animals comprising heterologous p28 gene and/or transgenic animals in which p28 is under the transcriptional control of a heterologous promoter.

In general, such a transgenic animal is typically an animal that has had appropriate p28 and/or other cytokine or cytokine receptor genes (or partial genes, e.g., comprising coding sequences coupled to a promoter) introduced into one or more of its cells artificially. This is most commonly done in one of two ways. First, a DNA encoding the relevant genes (or fragments thereof) can be integrated randomly by injecting it into the pronucleus of a fertilized ovum. In this case, the DNA can integrate anywhere in the genome. In this approach, there is no need for homology between the injected DNA and the host genome. Second, targeted insertion can be accomplished by introducing the (heterologous) DNA into embryonic stem (ES) cells and selecting for cells in which the heterologous DNA has undergone homologous recombination with homologous sequences of the cellular genome. Typically, there are several kilobases of homology between the heterologous and genomic DNA, and positive selectable markers (e.g., antibiotic resistance genes) are included in the heterologous DNA to provide for selection of transformants. In addition, negative selectable markers (e.g., “toxic” genes such as barnase) can be used to select against cells that have incorporated DNA by non-homologous recombination (i.e., random insertion).

One common use of targeted insertion of DNA is to make knock-out mice. Typically, homologous recombination is used to insert a selectable gene driven by a constitutive promoter into an essential exon of the gene that one wishes to disrupt (e.g., the first coding exon). To accomplish this, the selectable marker is flanked by large stretches of DNA that match the genomic sequences surrounding the desired insertion point. Once this construct is electroporated into ES cells, the cells' own machinery performs the homologous recombination. To make it possible to select against ES cells that incorporate DNA by non-homologous recombination, it is common for targeting constructs to include a negatively selectable gene outside the region intended to undergo recombination (typically the gene is cloned adjacent to the shorter of the two regions of genomic homology). Because DNA lying outside the regions of genomic homology is lost during homologous recombination, cells undergoing homologous recombination cannot be selected against, whereas cells undergoing random integration of DNA often can. A commonly used gene for negative selection is the herpes virus thymidine kinase gene, which confers sensitivity to the drug gancyclovir.

Following positive selection and negative selection if desired, ES cell clones are screened for incorporation of the construct into the correct genomic locus. Typically, one designs a targeting construct so that a band normally seen on a Southern blot or following PCR amplification becomes replaced by a band of a predicted size when homologous recombination occurs. Since ES cells are diploid, only one allele is usually altered by the recombination event so, when appropriate targeting has occurred, one usually sees bands representing both wild type and targeted alleles.

The embryonic stem (ES) cells that are used for targeted insertion are derived from the inner cell masses of blastocysts (early mouse embryos). These cells are pluripotent, meaning they can develop into any type of tissue.

Once positive ES clones have been grown up and frozen, the production of transgenic animals can begin. Donor females are mated, blastocysts are harvested, and several ES cells are injected into each blastocyst. Blastocysts are then implanted into a uterine horn of each recipient. By choosing an appropriate donor strain, the detection of chimeric offspring (i.e., those in which some fraction of tissue is derived from the transgenic ES cells) can be as simple as observing hair and/or eye color. If the transgenic ES cells do not contribute to the germline (sperm or eggs), the transgene cannot be passed on to offspring.

Further Details Regarding Cells Comprising Transgenic p28 and/or Modified p28 Genes

One feature of the invention is the production of recombinant cells, e.g., expressing a heterologous p28 or modified p28 gene, e.g., modified to express a p28 variant. Co-expression in a recombinant cell is particularly useful when screening for modulators of p28 activity. By co-expressing p28 from a therapeutically relevant target (such as a human cell) along with one or more putative modulators of a p28 activity, it is possible to appropriately screen for activity in a model cell.

In these recombinant cell embodiments, the biological sample to be tested is derived from the recombinant cell, which is selected, e.g., for ease of culture and manipulation. The cells can be, e.g., human, rodent, insect, Xenopus, etc. and will typically be a cell in culture (or an oocyte in the case of Xenopus).

p28 or modified p28 nucleic acids (e.g., which express p28 polypeptide variants) are typically introduced into cells in cloning and/or expression vectors to facilitate introduction of the nucleic acid and expression of encoded proteins. Vectors can include, e.g., plasmids, cosmids, viruses, YACs, bacteria, poly-lysine, etc. A “vector nucleic acid” is a nucleic acid molecule into which a heterologous nucleic acid is optionally inserted that can then be introduced into an appropriate host cell. Vectors preferably have one or more origins of replication, and one or more sites into which the recombinant DNA can be inserted. Vectors often have convenient means by which cells with vectors can be selected from those without, e.g., they encode drug resistance genes. Common vectors include plasmids, viral genomes, and (e.g., in yeast and bacteria) artificial chromosomes. “Expression vectors” are vectors that comprise elements that provide for or facilitate the transcription of nucleic acids that are cloned into such vectors. Such elements can include, e.g., promoters and/or enhancers operably coupled to a nucleic acid of interest.

In general, appropriate expression vectors are known in the art. For example, pET-14b, pCDNA1Amp, and pVL1392 are available from Novagen and Invitrogen and are suitable vectors for expression in E. coli, COS cells and baculovirus infected insect cells, respectively. pcDNA-3, pEAK, and vectors that permit the generation of PKD2L1 RNA for in vitro and in vivo expression experiments (e.g., in vitro translations and Xenopus oocyte injections) are also useful. These vectors are simply illustrative of those that are known in the art, with thousands of suitable vectors being available. Suitable host cells can be, e.g., any cell capable of growth in a suitable media and allowing purification of an expressed protein. Examples of suitable host cells include bacterial cells, such as E. coli, Streptococci, Staphylococci, Streptomyces and Bacillus subtilis cells; fungal cells such as yeast cells, Pichia, and Aspergillus cells; insect cells such as Drosophila S2 and Spodoptera Sf9 cells, mammalian cells such as CHO, COS, and HeLa; and even plant cells.

Cells are transformed with relevant genes (p28, a modified p28 gene that encodes a p28 polypeptide variant, etc.) according to standard cloning and transformation methods. Such genes can also be isolated from resulting recombinant cells using standard methods. General texts which describe molecular biological techniques for making nucleic acids, including the use of vectors, promoters and many other relevant topics, include Berger and Kimmel, Guide to Molecular Cloning Techniques, Methods in Enzymology volume 152 Academic Press, Inc., San Diego, Calif. (Berger); Sambrook; Ausubel; Kauman; and Rapley (above).

In addition, a plethora of kits are commercially available for the preparation, purification and cloning of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms, or the like.

As noted, typical vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Gillam & Smith (1979) “Site-specific mutagenesis using synthetic oligodeoxyribonucleotide primers: I. Optimum conditions and minimum oligodeoxyribonucleotide length.” Gene 8: 81-97; Roberts et al. (1987) “Generation of an antibody with enhanced affinity and specificity for its antigen by protein engineering.” Nature 328: 731-734; Schneider et al. (1995) “Functional Purification of a Bacterial ATP-Binding Cassette Transporter Protein (MalK) from the Cytoplasmic Fraction of an Overproducing Strain.” Protein Expr. Purif. 6435: 10-14; Ausubel, Sambrook, and Berger (above). A catalogue of Bacteria and Bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

In addition, essentially any nucleic acid (and virtually any labeled nucleic acid, whether standard or non-standard) can be custom or standard ordered from any of a variety of commercial sources, such as The Midland Certified Reagent Company (mcrc@oligos.com), The Great American Gene Company (www.genco.com), ExpressGen Inc. (www.expressgen.com), Operon Technologies Inc. (Alameda, Calif.) and many others.

Other useful references, e.g., for cell isolation and culture (e.g., for subsequent nucleic acid isolation) include Freshney (2005) Culture of Animal Cells, a Manual of Basic Technique, fifth edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York); and Atlas and Parks (eds) The Handbook of Microbiological Media (1993) CRC Press, Boca Raton, Fla.

### Nucleic Acid and Polypeptide Sequences and Variants

Sequences for a variety of naturally occurring WSX-1, gp130, p28, and EBI3 proteins and nucleic acids are publicly available. See, for example, protein sequence id NP_{—}663634 and nucleotide sequence accession number NM_{—}145659 for human p28, protein sequence id NP_{—}005746 and nucleotide sequence accession number NM_{—}005755 for human EBI3, protein sequence id NP_{—}004834 and nucleotide sequence accession number NM_{—}004843 for human WSX-1, protein sequence id NP_{—}002175 and nucleotide sequence accession number NM_{—}002184 for human gp130, protein sequence id NP_{—}663611.1 and nucleotide sequence accession number NM_{—}145636.1 for murine p28, protein sequence id NP_{—}056581.1 and nucleotide sequence accession number NM_{—}015766 for mouse EBI3, protein sequence id NP_{—}057880.1 and nucleotide sequence accession number NM_{—}016671 for mouse WSX-1, and protein sequence id NP_{—}034690 and nucleotide sequence accession number NM_{—}010560 for mouse gp130. Sequences homologous or substantially identical to these nucleotide or amino acid sequences are also of interest in the present invention. As noted herein, various soluble and/or fusion variants of such proteins have been described (see, e.g., U.S. patent application publication 20040185049 and Wirtz et al., supra), and recombinant varieties of p28 and EBI3 are commercially available.

One of skill will appreciate that the invention provides many related sequences with the functions described herein, for example, polynucleotides encoding p28 or a p28 fusion protein, or variants thereof.

Because of the degeneracy of the genetic code, many polynucleotides equivalently encode a given polypeptide sequence. Polynucleotide sequences complementary to any of the above described sequences are included among the polynucleotides of the invention. Similarly, an artificial or recombinant nucleic acid that hybridizes to a polynucleotide indicated above under highly stringent conditions over substantially the entire length of the nucleic acid (and is other than a naturally occurring polynucleotide) is a polynucleotide of the invention.

In certain embodiments, a vector (e.g., a plasmid, a cosmid, a phage, a virus, etc.) comprises a polynucleotide of the invention. In one embodiment, the vector is an expression vector. In another embodiment, the expression vector includes a promoter operably linked to one or more of the polynucleotides of the invention. In another embodiment, a cell comprises a vector that includes a polynucleotide of the invention.

One of skill will also appreciate that many variants of p28 are included in the invention. For example, conservative variations of the disclosed sequences that yield a functionally similar sequence are included in the invention. Variants of the nucleic acid polynucleotide sequences, wherein the variants hybridize to at least one disclosed sequence, are considered to be included in the invention. Unique subsequences of the sequences disclosed herein, as determined by, e.g., standard sequence comparison techniques, are also included in the invention.

Conservative Variations

Owing to the degeneracy of the genetic code, “silent substitutions” (i.e., substitutions in a nucleic acid sequence which do not result in an alteration in an encoded polypeptide) are an implied feature of every nucleic acid sequence that encodes an amino acid sequence. Similarly, “conservative amino acid substitutions,” where one or a limited number of amino acids in an amino acid sequence are substituted with different amino acids with highly similar properties, are also readily identified as being highly similar to a disclosed construct. Such conservative variations of each disclosed sequence are a feature of the present invention.

“Conservative variations” of a particular nucleic acid sequence refers to those nucleic acids that encode identical or essentially identical amino acid sequences, or, where the nucleic acid does not encode an amino acid sequence, to essentially identical sequences. One of skill will recognize that individual substitutions, deletions or additions which alter, add or delete a single amino acid or a small percentage of amino acids (typically less than 5%, more typically less than 4%, 2% or 1%) in an encoded sequence are “conservatively modified variations” where the alterations result in the deletion of an amino acid, addition of an amino acid, or substitution of an amino acid with a chemically similar amino acid, while retaining the relevant function. Thus, “conservative variations” of a listed polypeptide sequence of the present invention include substitutions of a small percentage, typically less than 5%, more typically less than 2% or 1%, of the amino acids of the polypeptide sequence, with an amino acid of the same conservative substitution group. Finally, the addition of sequences that do not alter the encoded activity of a nucleic acid molecule, such as the addition of a non-functional or tagging sequence (introns in the nucleic acid, poly His or similar sequences in the encoded polypeptide, etc.), is a conservative variation of the basic nucleic acid or polypeptide.

Conservative substitution tables providing functionally similar amino acids are well known in the art, where one amino acid residue is substituted for another amino acid residue having similar chemical properties (e.g., aromatic side chains or positively charged side chains), and therefore does not substantially change the functional properties of the polypeptide molecule. Table 1 sets forth example groups that contain natural amino acids of like chemical properties, where substitutions within a group is a “conservative substitution”.

TABLE 1
Conservative Amino Acid Substitutions

| | | | Positively | Negatively |
|---|---|---|---|---|
| Nonpolar and/or | Polar, | Aromatic | Charged | Charged |
| Aliphatic Side | Uncharged | Side | Side | Side |
| Chains | Side Chains | Chains | Chains | Chains |
| Glycine | Serine | Phenylalanine | Lysine | Aspartate |
| Alanine | Threonine | Tyrosine | Arginine | Glutamate |
| Valine | Cysteine | Tryptophan | Histidine | |
| Leucine | Methionine | | | |
| Isoleucine | Asparagine | | | |
| Proline | Glutamine | | | |

Sequence Comparison, Identity, and Homology

The terms “identical” or “percent identity,” in the context of two or more nucleic acid or polypeptide sequences, refer to two or more sequences or subsequences that are the same or have a specified percentage of amino acid residues or nucleotides that are the same, when compared and aligned for maximum correspondence, as measured using one of the sequence comparison algorithms described below (or other algorithms available to persons of skill) or by visual inspection.

The phrase “substantially identical,” in the context of two nucleic acids or polypeptides (e.g., DNAs encoding a p28 polypeptide, or the amino acid sequence of a p28 polypeptide) refers to two or more sequences or subsequences that have at least about 60%, about 80%, about 90%, about 95%, about 98%, about 99% or more nucleotide or amino acid residue identity, when compared and aligned for maximum correspondence, as measured using a sequence comparison algorithm or by visual inspection. Such “substantially identical” sequences are typically considered to be “homologous,” without reference to actual ancestry. Preferably, the “substantial identity” exists over a region of the sequences that is at least about 50 residues in length, more preferably over a region of at least about 100 residues, and most preferably, the sequences are substantially identical over at least about 150 residues, or over the full length of the two sequences to be compared.

Proteins and/or protein sequences are “homologous” when they are derived, naturally or artificially, from a common ancestral protein or protein sequence. Similarly, nucleic acids and/or nucleic acid sequences are homologous when they are derived, naturally or artificially, from a common ancestral nucleic acid or nucleic acid sequence. Homology is generally inferred from sequence similarity between two or more nucleic acids or proteins (or sequences thereof). The precise percentage of similarity between sequences that is useful in establishing homology varies with the nucleic acid and protein at issue, but as little as 25% sequence similarity over 50, 100, 150 or more residues (nucleotides or amino acids) is routinely used to establish homology. Higher levels of sequence similarity, e.g., 30%, 40%, 50%, 60%, 70%, 80%, 90%, 95%, or 99% or more, can also be used to establish homology. Methods for determining sequence similarity percentages (e.g., BLASTP and BLASTN using default parameters) are described herein and are generally available. “Orthologs” are genes in different species that evolved from a common ancestral gene by speciation. Normally, orthologs retain the same or similar function in the course of evolution. As used herein “orthologs” are included in the term “homologs.”

For sequence comparison and homology determination, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Current Protocols in Molecular Biology, Ausubel et al., eds., Current Protocols, a joint venture between Greene Publishing Associates, Inc. and John Wiley & Sons, Inc., supplemented through 2007).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul, Proc. Natl. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### Making and Isolating Recombinant Polypeptides

Generally, nucleic acids encoding a polypeptide of the invention or for use in the methods or compositions of the invention can be made by cloning, recombination, in vitro synthesis, in vitro amplification and/or other available methods. Essentially any nucleic acid can be custom or standard ordered from any of a variety of commercial sources, such as Operon Technologies Inc. (Alameda, Calif.). In addition, a variety of recombinant methods can be used for expressing an expression vector that encodes a polypeptide of the invention. Recombinant methods for making nucleic acids, expression and isolation of expressed products are well known and are described, e.g., in Sambrook, Ausubel, and Innis et al. (eds.), PCR Protocols: A Guide to Methods and Applications, Academic Press Inc., San Diego, Calif. (1990).

A plethora of kits are commercially available for the purification of plasmids or other relevant nucleic acids from cells, (see, e.g., EasyPrep™, FlexiPrep™, both from Pharmacia Biotech; StrataClean™, from Stratagene; and, QIAprep™ from Qiagen). Any isolated and/or purified nucleic acid can be further manipulated to produce other nucleic acids, used to transfect cells, incorporated into related vectors to infect organisms for expression, and/or the like. Typical cloning vectors contain transcription and translation terminators, transcription and translation initiation sequences, and promoters useful for regulation of the expression of the particular target nucleic acid. The vectors optionally comprise generic expression cassettes containing at least one independent terminator sequence, sequences permitting replication of the cassette in eukaryotes, or prokaryotes, or both, (e.g., shuttle vectors) and selection markers for both prokaryotic and eukaryotic systems. Vectors are suitable for replication and integration in prokaryotes, eukaryotes, or both. See, Giliman & Smith, Gene 8:81 (1979); Roberts, et al., Nature, 328:731 (1987); Schneider, B., et al., Protein Expr. Purif. 6435:10 (1995); Ausubel supra, Sambrook supra, and Berger and Kimmel, Guide to Molecular Cloning Techniques, Methods in Enzymology volume 152 Academic Press, Inc., San Diego, Calif. A catalogue of bacteria and bacteriophages useful for cloning is provided, e.g., by the ATCC, e.g., The ATCC Catalogue of Bacteria and Bacteriophage published yearly by the ATCC. Additional basic procedures for sequencing, cloning and other aspects of molecular biology and underlying theoretical considerations are also found in Watson et al. (1992) Recombinant DNA Second Edition, Scientific American Books, NY.

Other useful references, e.g. for cell isolation and culture (e.g., for subsequent nucleic acid or polypeptide isolation) include Freshney (1994) Culture of Animal Cells, a Manual of Basic Technique, third edition, Wiley-Liss, New York and the references cited therein; Payne et al. (1992) Plant Cell and Tissue Culture in Liquid Systems John Wiley & Sons, Inc. New York, N.Y.; Gamborg and Phillips (eds) (1995) Plant Cell, Tissue and Organ Culture; Fundamental Methods Springer Lab Manual, Springer-Verlag (Berlin Heidelberg New York) and Atlas and Parks (eds) The Handbook of Microbiological Media (1993) CRC Press, Boca Raton, Fla.

A variety of protein isolation and detection methods are known and can be used to isolate polypeptides, e.g., from recombinant cultures of cells expressing the recombinant fusion or soluble proteins of the invention. A variety of protein isolation and detection methods are well known in the art, including, e.g., those set forth in R. Scopes, Protein Purification, Springer-Verlag, N.Y. (1982); Deutscher, Methods in Enzymology Vol. 182: Guide to Protein Purification, Academic Press, Inc. N.Y. (1990); Sandana (1997) Bioseparation of Proteins, Academic Press, Inc.; Bollag et al. (1996) Protein Methods, 2^{nd} Edition Wiley-Liss, NY; Walker (1996) The Protein Protocols Handbook Humana Press, NJ, Harris and Angal (1990) Protein Purification Applications: A Practical Approach IRL Press at Oxford, Oxford, England; Harris and Angal Protein Purification Methods: A Practical Approach IRL Press at Oxford, Oxford, England; Scopes (1993) Protein Purification: Principles and Practice 3^{rd} Edition Springer Verlag, NY; Janson and Ryden (1998) Protein Purification: Principles, High Resolution Methods and Applications, Second Edition Wiley-VCH, NY; and Walker (1998) Protein Protocols on CD-ROM Humana Press, NJ; and the references cited therein. Additional details regarding protein purification and detection methods can be found in Satinder Ahuja ed., Handbook of Bioseparations, Academic Press (2000).

p28 polypeptides and variants thereof can be expressed and purified by one of skill. Alternatively, a number of such polypeptides are commercially available. For example, recombinant p28 and EBI3 are available from Abnova Corporation (www (dot) abnova (dot) corn (dot) tw). Where polypeptide complexes are desired, the two (or more) polypeptide components of the complex are optionally co-expressed and purified together as a complex, or the components can be purified separately and then combined to form the complex. The components are optionally noncovalently associated in the complex, or are optionally covalently connected by a chemical crosslinker or the like in the complex. In the present invention, the p28 polypeptides (or p28 polypeptide variants) are preferably not in a complex with EBI3, gp130, or WSX-1

### EXAMPLE

The following example is offered to illustrate, but not limit, the claimed invention. It is understood that the examples and embodiments described herein are for illustrative purposes only and that various modifications or changes in light thereof will be suggested to persons skilled in the art and are to be included within the spirit and purview of this application and scope of the appended claims. One of skill will recognize a variety of non-critical parameters that can be modified to achieve essentially similar results.

Overview: IL-27p28 Antagonizes gp130-Mediated Cytokine Signaling

The cytokines IL-6, IL-12, IL-23 and IL-27 are closely related to one another based on similarities of their structural motifs, a common four-helix bundle, and their shared usage of various receptor subunits. These type I cytokines initiate their activity through membrane bound receptor complexes that include either gp130 or Th-12Rβ1 in order to influence the development and regulation of inflammatory responses (Kastelein et al. (2007) “Discovery and biology of IL-23 and IL-27: related but functionally distinct regulators of inflammation.” Annu Rev Immunol 25: 221-242). These cytokines have received a lot of recent attention due to their ability to direct T_{H}1 and T_{H}17 responses as well as the ability of IL-27 to regulate these responses. IL-6, the prototypical member of this family is a single subunit cytokine that binds to gp130 and a unique surface bound IL-6Rα chain. A soluble version of the IL-6R can form a complex with IL-6, which can then bind gp130 and transduce a signal through a process termed trans-signaling (Jones (2005) “Directing transition from innate to acquired immunity: defining a role for IL-6.” J Immunol 175: 3463-3468; Jones et al. (2005) “IL-6 transsignaling: the in vivo consequences.” J Interferon Cytokine Res 25: 241-253). Recent reports have indicated that this latter process has been implicated in the control of leukocyte recruitment, activation and apoptotic clearance in a number of chronic inflammatory diseases such as inflammatory bowel disease, peritonitis, rheumatoid arthritis and asthma (Jones (2005) “Directing transition from innate to acquired immunity: defining a role for IL-6.” J Immunol 175: 3463-3468; Jones et al. (2005) “IL-6 transsignaling: the in vivo consequences.” J Interferon Cytokine Res 25: 241-253).

IL-27 is a heterodimeric cytokine composed of p28 and EBI3 (Pflanz et al. (2002) “IL-27, a heterodimeric cytokine composed of EBI3 and p28 protein, induces proliferation of naive CD4(+) T cells.” Immunity 16: 779-790). While p28 is a four-helix bundle protein similar to IL-6 the structure of EBI3 resembles that of the sIL-6R. Unlike IL-6, IL-27 employs a unique receptor subunit IL-27ra (also known as WSX-1 or TCCR) to pair with gp130 for signaling (Pflanz et al. (2002) “IL-27, a heterodimeric cytokine composed of EBI3 and p28 protein, induces proliferation of naive CD4(+) T cells.” Immunity 16: 779-790; Pflanz et al. (2004) “WSX-1 and glycoprotein 130 constitute a signal-transducing receptor for IL-27.” J Immunol 172: 2225-2231). Whereas a disulfide bond links the individual subunits of the other heterodimeric cytokines of this family, IL-12 and IL-23, the subunits of IL-27 do not interact in this manner suggesting an alternative mechanism of folding and assembly for IL-27 (Batten et al. (2007) “The biology and therapeutic potential of interleukin 27.” J Mol Med 85: 661-672). Furthermore, a difference in transcriptional regulation of p28 and EBI3 can result in the secretion of these individual subunits. Thus, it is possible that the p28 and EBI3 subunits of IL-27 can be secreted independently from the other, thus allowing for extracellular association or pairing of each subunit with itself or other proteins. Previous work from this laboratory has shown that purified p28 was capable of suppressing IL-17 production by CD4⁺ T cells grown under Th17 polarizing conditions in vitro suggesting that p28 has biological activity, or that EBI3 was present in the culture conditions to form heterodimers (Stumhofer et al. (2006) “Interleukin 27 negatively regulates the development of interleukin 17-producing T helper cells during chronic inflammation of the central nervous system.” Nat Immunol 7: 937-945). The studies reported here indicate that while p28 by itself does not activate STAT-signaling pathways it can function as a natural antagonist of IL-6 and 1L-27 signaling in vitro.

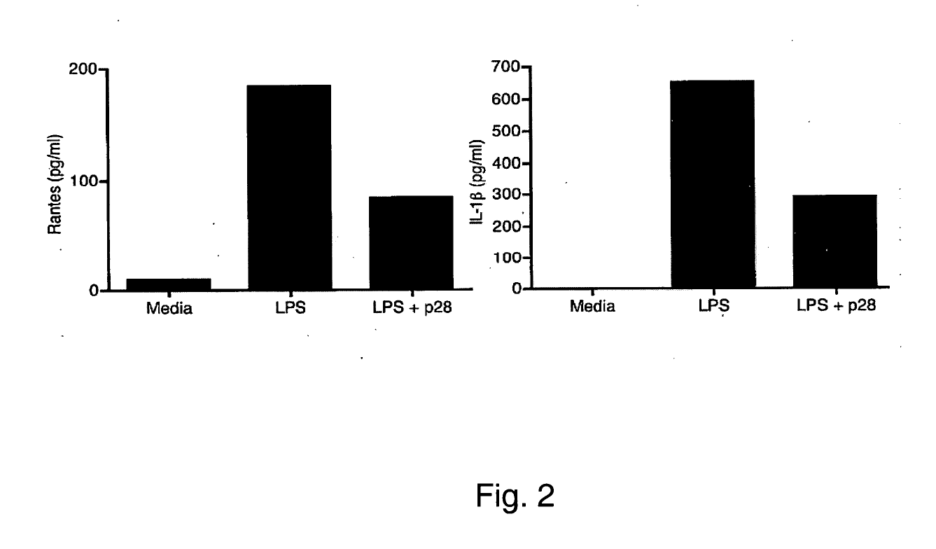

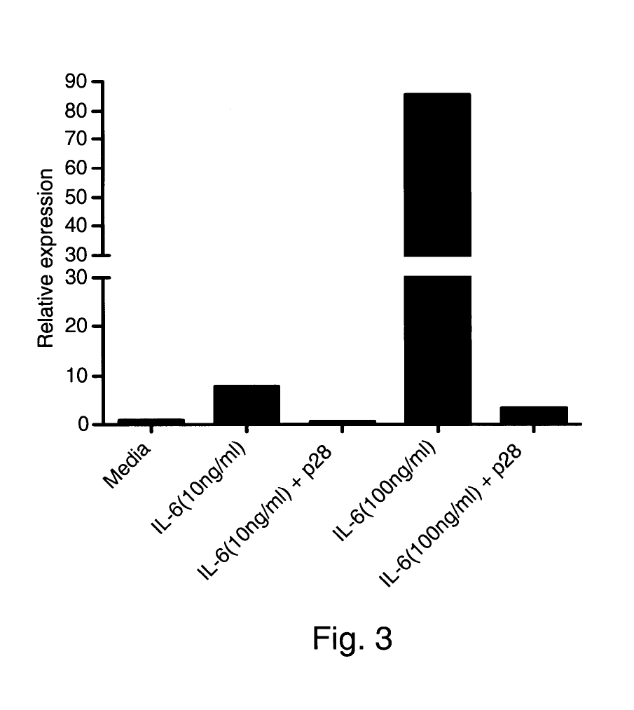

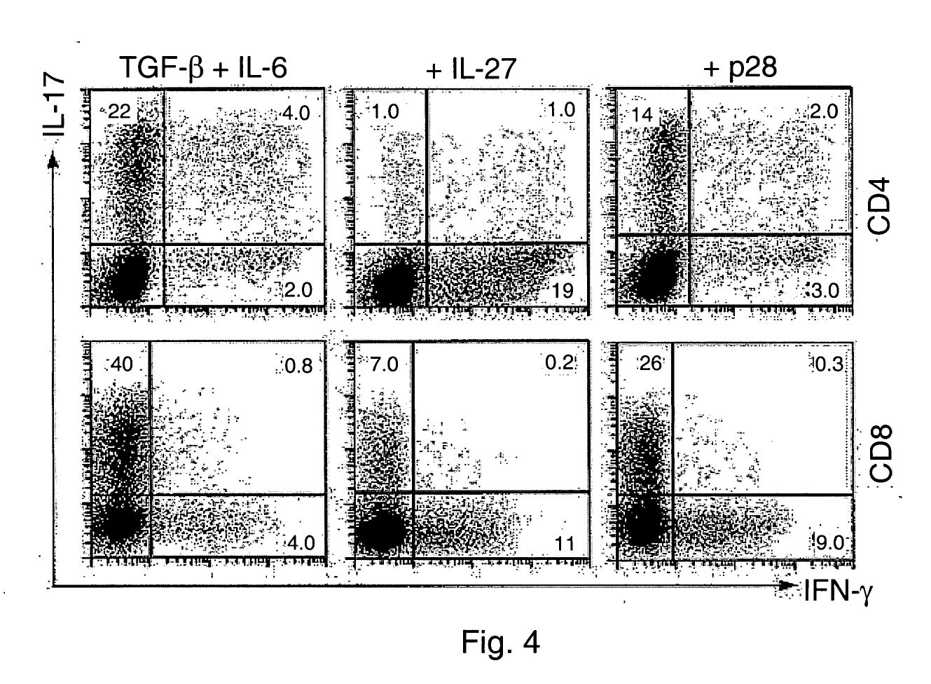

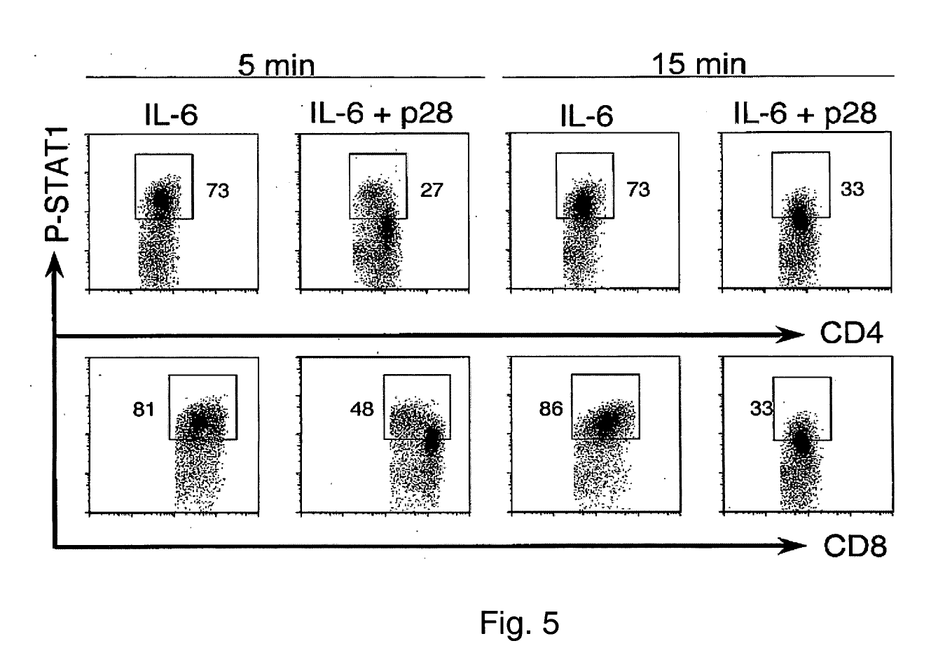

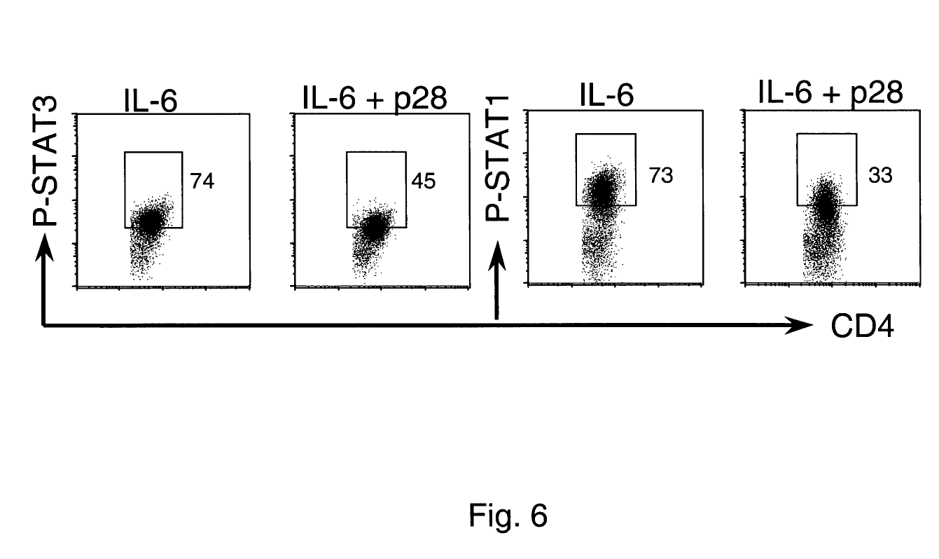

FIGS. 2-6 provide the first evidence that p28 alone can be used to suppress IL-6 mediated signaling and can thus be used to suppress, e.g., an IL-6-associated disease or inflammatory response.

### EBI3 is Not Required for the Secretion of p28 in Response to Microbial Stimuli

To further evaluate a physiological role for p28, it was of interest to determine if p28 can be secreted independently of EBI3 by endogenous cells. Therefore, bone-marrow derived macrophages (BMMφs) and dendritic cells (BMDCs) from wild-type and EBI3^{-/-} mice were stimulated with LPS, IFN-γ or the combination for 24 h, and the ability of these cells to secrete p28 was determined using a sandwich ELISA that specifically recognizes IL-27p28. EBI3−/− mice on a C57BL/6 background were provided by M. Elloso (Centocor). Wild-type C57BL/6 mice were purchased from Jackson laboratories. Mice were housed and bred in specific pathogen-free facilities in the Department of Pathobiology at the University of Pennsylvania in accordance with institutional guidelines.

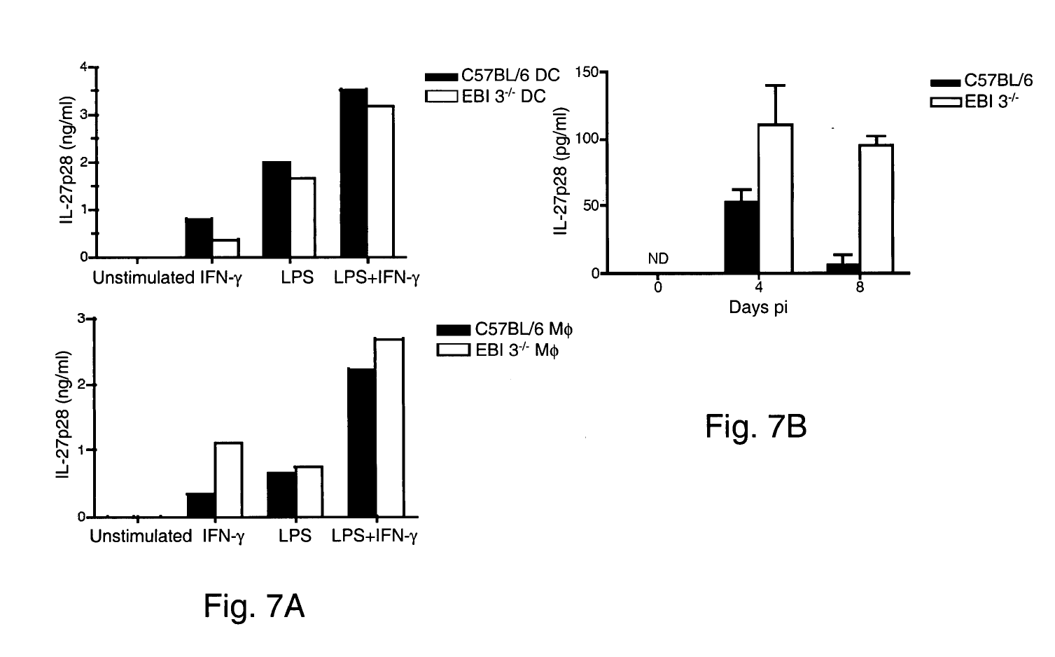

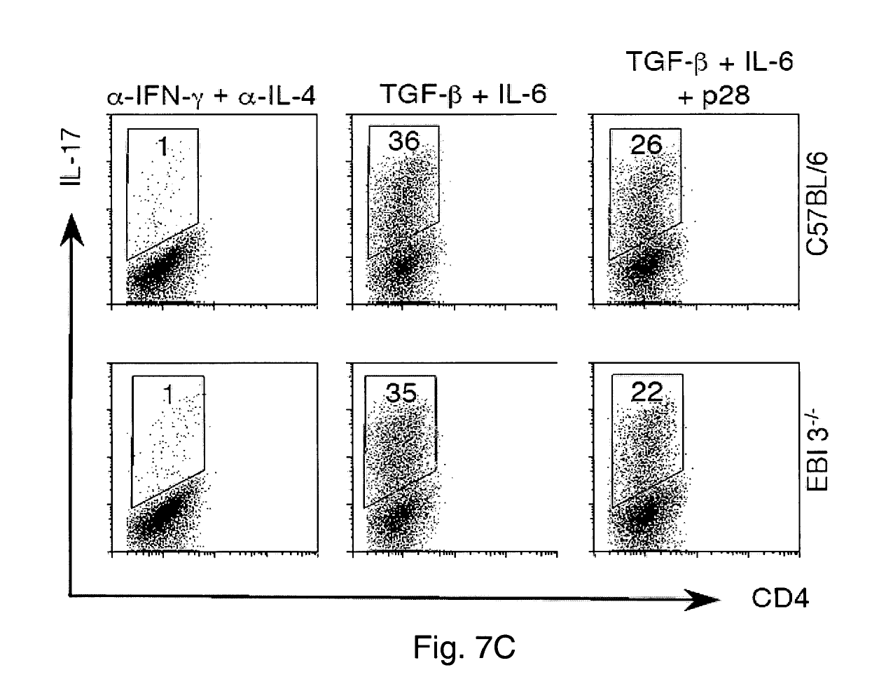

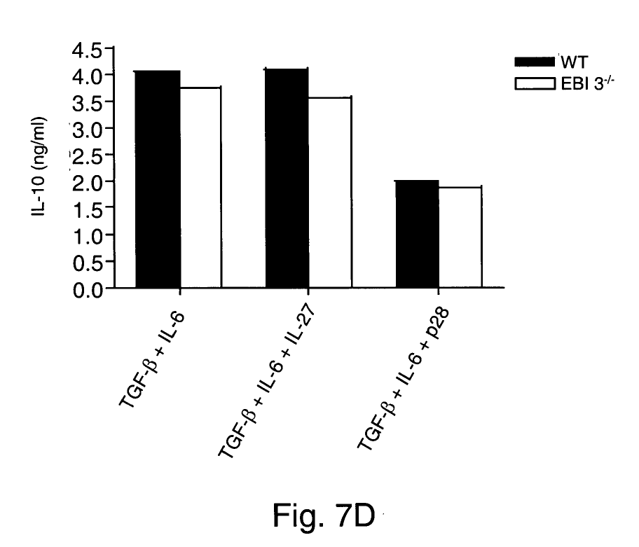

As previously reported (Liu et al. (2007) “Regulation of IL-27 p28 gene expression in macrophages through MyD88- and interferon-gamma-mediated pathways.” J Exp Med 204: 141-152; Molle et al. (2007) “IL-27 synthesis induced by TLR ligation critically depends on IFN regulatory factor 3.” J Immunol 178: 7607-7615) LPS and IFN-γ induced p28 secretion by wild-type BMMφs and BMDCs (FIG. 7a). In addition, these stimuli also resulted in equivalent levels of p28 production by EBI3^{-/-} BMMφs and BMDCs with the highest level of p28 secretion by both cell types occurring with LPS and IFN-γ costimulation. Similar results were seen with other TLR agonists including CpG. Furthermore, not only were measurable levels of IL-27p28 detected in the serum of wild-type and EBI3^{-/-} mice during acute infection with Toxoplasma gondii, but there were higher and more sustained levels of IL-27p28 present in the blood of EBI3^{-/-} mice (FIG. 7b). Together these results indicate that p28 can be secreted efficiently in the absence of EBI3 and suggests a potential biological role for this subunit in vivo.

### p28 is Biologically Active in the Absence of EBI3

Previous studies from this laboratory reported that recombinant p28 modestly, but consistently, suppressed IL-17 production under conditions using TGF-β plus IL-6 to induce Th17 development (Stumhofer et al. (2006) “Interleukin 27 negatively regulates the development of interleukin 17-producing T helper cells during chronic inflammation of the central nervous system.” Nat Immunol 7: 937-945). However, it was unclear whether exogenously added p28 was capable of binding to soluble EBI3 present in the supernatant. In order to determine if p28 can limit IL-17 production as a single subunit under these same conditions, splenocytes from EBI3^{-/-} mice were used. Similar to the results seen with control wild-type splenocytes, p28 antagonized the production of IL-17 by EBI3 deficient CD4⁺ T cells under Th17 inducing conditions . In addition to promoting IL-17 production by CD4⁺ T cells, previous studies have shown that TGF-β and IL-6 also induce IL-10, and IL-27 had no effect on IL-10 production under these conditions (Stumhofer et al. (2007) “Interleukins 27 and 6 induce STAT3-mediated T cell production of interleukin 10.” Nat Immunol 8: 1363-1371; McGeachy et al. (2007) “TGF-beta and IL-6 drive the production of IL-17 and IL-10 by T cells and restrain T(H)-17 cell-mediated pathology.” Nat Immunol 8: 1390-1397). When p28 was added to the cultures with TGF-13 plus IL-6, it not only reduced the capacity of wild-type and EBI3^{-/-} CD4⁺ T cells to make IL-17, but it also limited IL-10 production (FIG. 7d). Also, while IL-27 by itself or in synergy with TGF-β has been found to promote IL-10 production by CD4+ T cells (Stumhofer et al. (2007) “Interleukins 27 and 6 induce STAT3-mediated T cell production of interleukin 10.” Nat Immunol 8: 1363-1371), p28 alone or in the presence of TGF-β did not support the development of IL-10 producing T cells. Thus, these results indicate that p28 does not possess the same biological activity as IL-27 in these in vitro assays.

### p28 Antagonizes IL-6 and IL-27 STAT Signaling

Since IL-27p28 and IL-6 both form a four alpha-helix bundle and IL-27 mediated signaling can antagonize the ability of IL-6 to promote Th17 differentiation it could be predicted that p28 alone could act in a manner analogous to IL-27 and induce STAT signaling. As IL-6 and IL-27 mediated signaling primarily activate STAT3 and STAT1, the ability of p28 to induce phosphorylation of these STAT proteins in purified T cells from C57BL/6 mice was measured over a period of one hour.

Recombinant IL-27p28 was produced as follows: The recombinant mouse IL-27p28 (rmp28) gene, Genbank accession number AY099297, was cloned from activated mouse macrophage cDNA via DNA primer extension. The forward DNA primer, ttcccaacagaccccctgagcc, and the reverse DNA primer, ttaggaatcccaggctgagcctg, were used to produce the mature 621 base pair mp28 DNA for expression in a proprietary E. coli expression system. The proprietary plasmid containing the mature fragment of the mp28 gene was confirmed via nucleotide sequencing and then transfected into competent DH5alpha E. coli for fermentation and inclusion body production. rmp28 inclusion bodies were collected from the bacteria and processed through a proprietary refolding platform.

Following folding, the protein was concentrated in a Millipore Cassette concentrator, molecular weight cut off of 3 kDa. The recombinant protein was then centrifuged in a Beckman J2-21 centrifuge for 45 minutes at 8,000rpm to remove any insoluble particulates. It was then carefully titrated to pH 6.0 and loaded onto a 20 ml Pharmacia Ion Exchange column and eluted with a salt gradiet from 0 to 1.0 M NaCl. Fractions were run on a 4-20% SDS-PAGE Tris/Glycine gel and pooled. The pool was dialyzed over night against 10 mM Tris, pH 8.0 buffer, 4 degrees. The next day the dialyzed pool was loaded onto an 80 micron hydroxylapetite column and a phosphate gradient from 2 mM to 70 mM NaP, pH 7.5 over 20 column volumes was run to elute the protein. Fractions were pooled based on purity and dialyzed over night at 4° C. against 10 mM NaP, pH 7.5. Protein was quantitated by A280, sterile filtered through a 0.2 micron filter and lyophilized.

As shown in FIG. 8a, IL-6 and IL-27 induced high levels of STAT1 and STAT3 phosphorylation in purified CD4⁺ T cells following a 15-minute incubation, while p28 alone did not induce phosphorylation of STAT1 or STAT3. This result was consistent with each time point (5, 30 and 60 min) measured. However, pre-incubation of p28 with the purified T cells 2 h prior to IL-6 or IL-27 stimulation resulted in reduced IL-6 and IL-27 mediated phosphorylation of STAT1 and STAT3 in CD4⁺ and CD8⁺ T cells (FIG. 8a) indicating that p28 may serve as a general antagonist of gp130-mediated STAT signaling. Moreover, when CD4⁺ T cells were incubated with IL-6 hyperkine, a fusion protein consisting of human IL-6 and the sIL-6Rα chain that only signals through gp130 (Fischer et al. (1997) “I. A bioactive designer cytokine for human hematopoietic progenitor cell expansion.” Nat Biotechnol 15: 142-145), phosphorylation of STAT1 and STAT3 was observed, and this signaling event could also be antagonized by the inclusion of IL-27p28 (FIG. 8b). This finding suggests that p28 inhibits IL-6 trans-signaling by binding to gp130, thus limiting the availability of this receptor subunit for binding to the IL-6 hyperkine (FIG. 8b).

### Overexpression of p28 Inhibits the Activity of IL-6 and IL-27 in Vitro

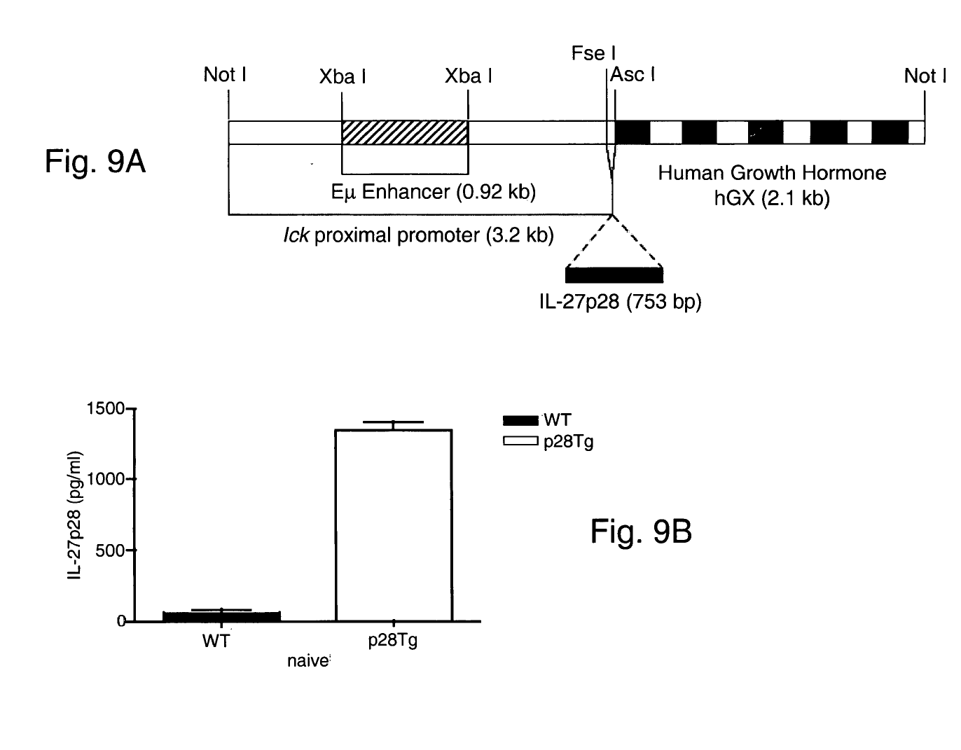

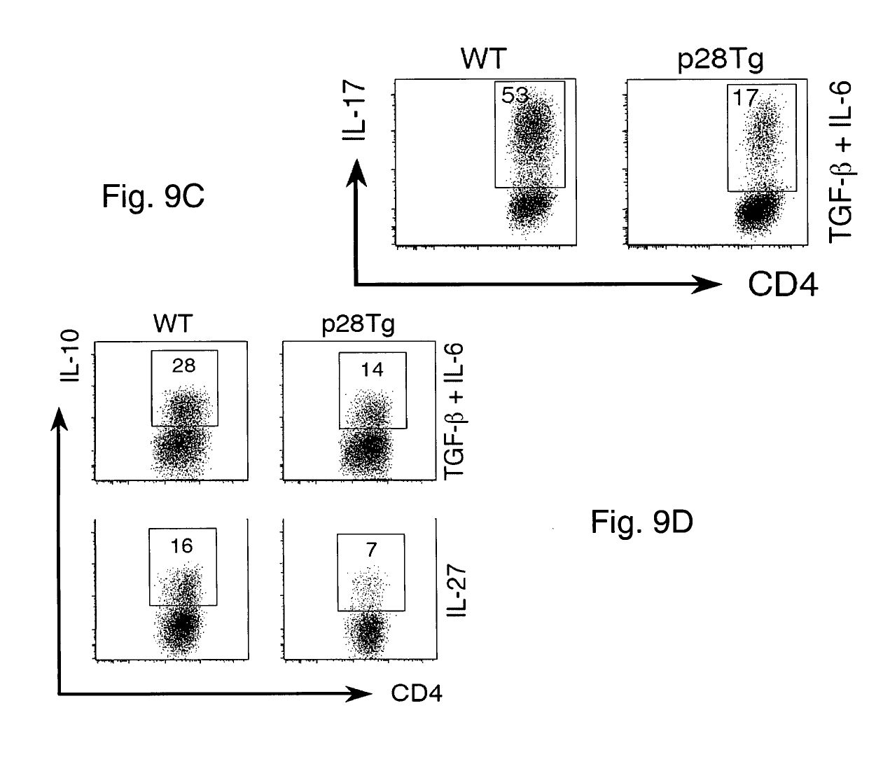

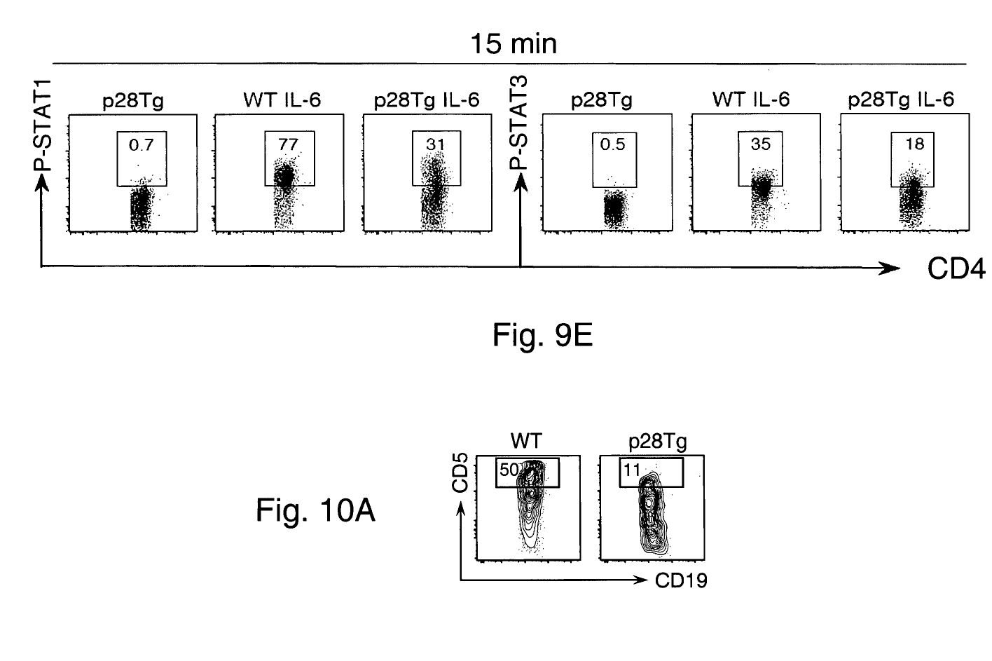

To examine the functional role of p28 in vivo, the murine p28 gene was cloned into a previously described expression vector downstream of a compound regulatory element in which the immunoglobulin intronic heavy chain enhancer (Eμ) and the lck proximal promoter are juxtaposed (Iritani et al. (1997) “Control of B cell development by Ras-mediated activation of Raf.” Embo J 16: 7019-7031) (FIG. 9a). Briefly, the open reading frames of mouse IL-27p28 was PCR amplified adding FseI-AscI sites. IL-27p28 cDNA (753bp) was then cloned into the Eμ lck transgene expression vector. This vector has been previously described (Iritani et al. (1997) “Control of B cell development by Ras-mediated activation of Raf.” EMBO J 16: 7019-7031) and includes the mouse lck proximal promoter and the mouse Eμ heavy chain enhancer. Expression is directed primarily to T and B cells as described (Iritani et al. (1997) “Control of B cell development by Ras-mediated activation of Raf.” EMBO J 16: 7019-7031). Expression cassettes were excised by NotI digestion and were microinjected into B6C3f1 murine oocytes fertilized by C57BL/6 males. Microinjection and production of transgenic mice followed procedures as described in Hogan, B. et al., Manipulating the Mouse Embryo, Cold Spring Harbor Laboratory Press (Hogan et al. (1994) Manipulating the Mouse Embryo (2nd edn. ed.). Cold Spring Habor Laboratory Press, Cold Spring Harbor, N.Y.). Transgenic founders were then bred to C57BL/6 mice to generate stable lines of transgenic mice expressing a single allele of p28. The p28 Tg mice were maintained by crossing with wild-type C57BL/6 mice from Jackson laboratories, and age and sex matched wild-type littermates were used as controls in all experiments. Confirmation of p28 expression by the transgenic mice was determined by measuring p28 in the sera using an IL-27p28 specific ELISA (R&D Systems). As shown in FIG. 9b, low basal levels of p28 were detected in the serum of naïve wild-type mice, but the level of IL-27p28 measured in the serum of naïve p28Tg mice was significantly higher than their wild-type littermates.

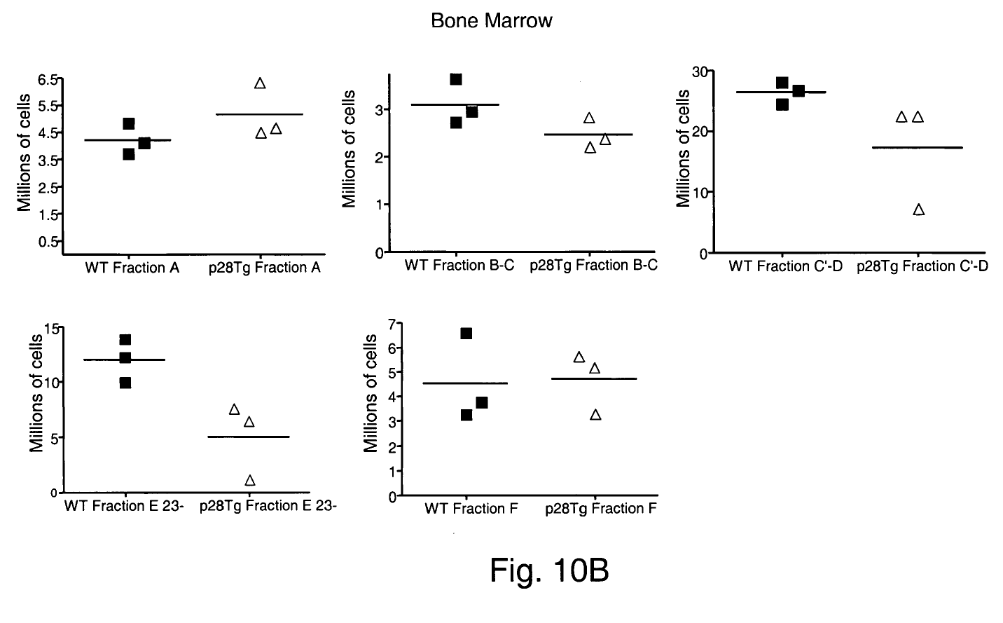

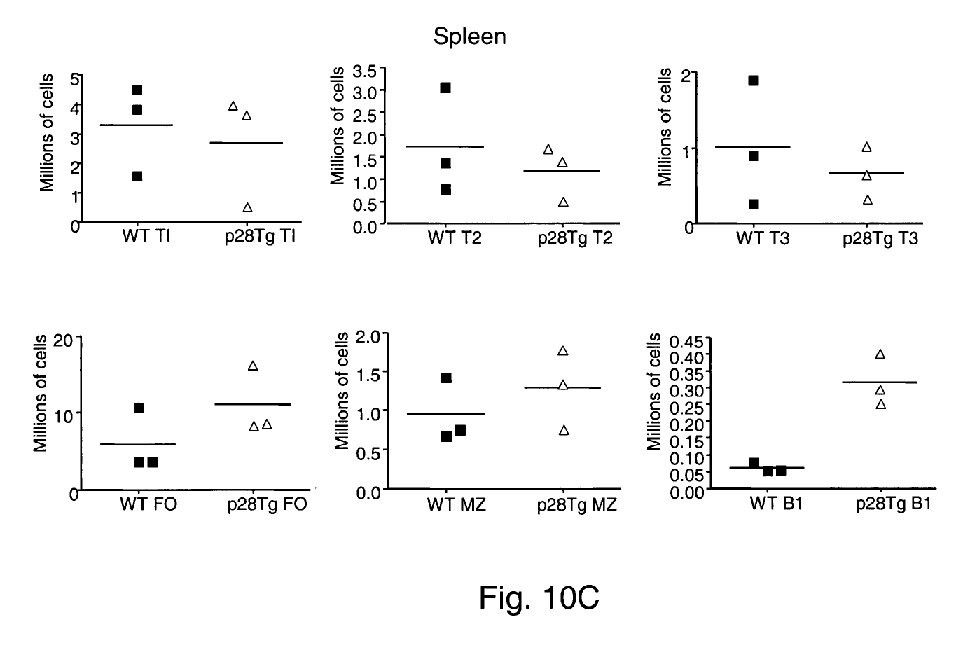

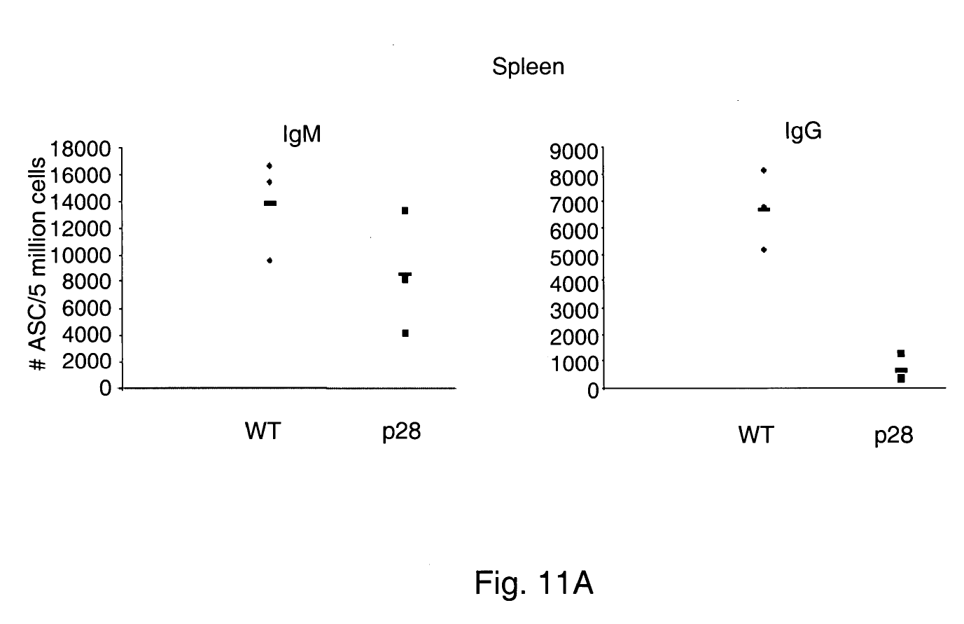

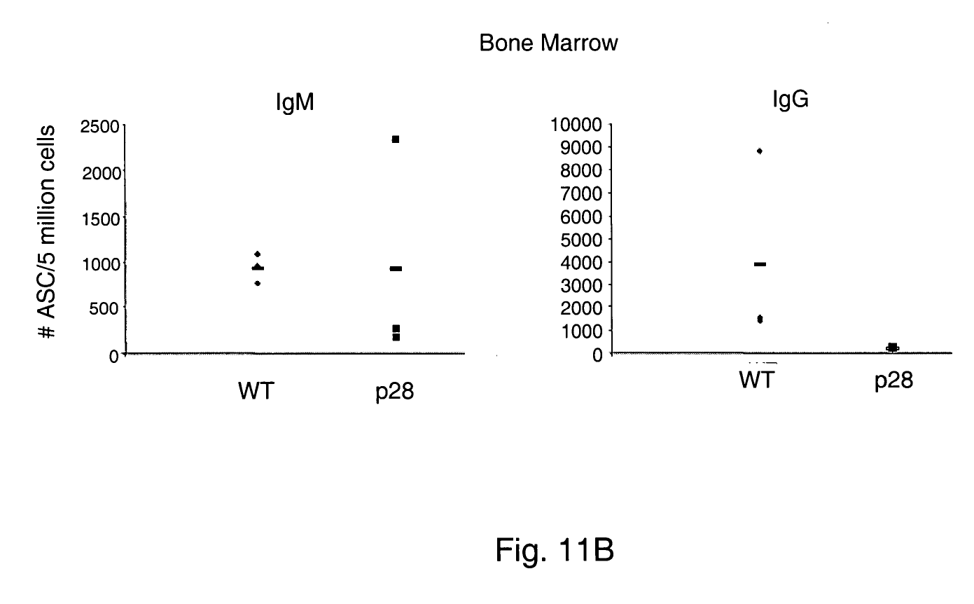

The transgenic mice show no developmental defects. Examination of the B cell compartment revealed that the p28Tg mice lack B-1a B cells in the peritoneum (FIG. 10a). While, no significant differences in the various stages of B-2 B cell development were observed within the bone marrow and spleen of p28Tg mice compared to their wild-type littermates (FIG. 10b, c), the number of IgG, but not IgM antibody-secreting cells were significantly reduced in these lymphoid compartments of naïve p28Tg mice (FIG. 11) suggesting that these mice have a defect in B cell differentiation. Furthermore, an assessment of the T cell compartment revealed an increase in the number of T cells in p28Tg mice compared to wild-type mice. However, constitutive expression of p28 did not result in any significant alteration in overall lymphoid development.

Next, the ability of IL-27p28 secreted by the transgenic mice to mirror the activity of recombinant p28 on CD4⁺ T cell activity in vitro was determined. CD4⁺ T cells were isolated from splenocyte samples and lymph nodes that were depleted of CD8⁺ and NK1.1⁺ cells to enrich for CD4⁺ T cells by magnetic bead separation (Polysciences). Cells were plated in 96-well round-bottom plates (Costar) at a density of 5×10⁶ cells per ml. Cells were stimulated with anti-CD3 (1 μg/ml; clone 145-2C11; eBioscience) and anti-CD28 (1 μg/ml; clone 37.51; eBioscience). For the production of IL-17+ T cells, cultures were supplemented with recombinant mouse M-6 (10 ng/ml; eBioscience) and human TGF-β1 (1 ng/ml; R&D Systems). Additionally, IFN-γ and IL-4 were neutralized with anti-IFN-γ (10 μg/ml; XMG1.2) and anti-IL-4 (10 μg/ml; 11B11; NCI Preclinical repository). In some cases IL-27 (50 ng/ml; Amgen) or p28 (100 ng/ml; Celtein) were added to the cultures. CD4⁺ T cells were supplemented with fresh medium and reagents on day 3 and were collected on day 4. T cells were then restimulated with PMA and ionomycin plus brefeldin A (Sigma). A FACSCalibar (BD Biosciences) or BDFACS CantoII (BD Biosciences) was used for flow cytometry, and the data were analyzed with FlowJo software (Treestar). For intracellular staining of GFP, cells were stained with polyclonal anti-GFP (14-6774-81; eBioscience), followed by a second stain with FITC-conjugated rat anti-rabbit (111-096-144; Jackson Immunoresearch).

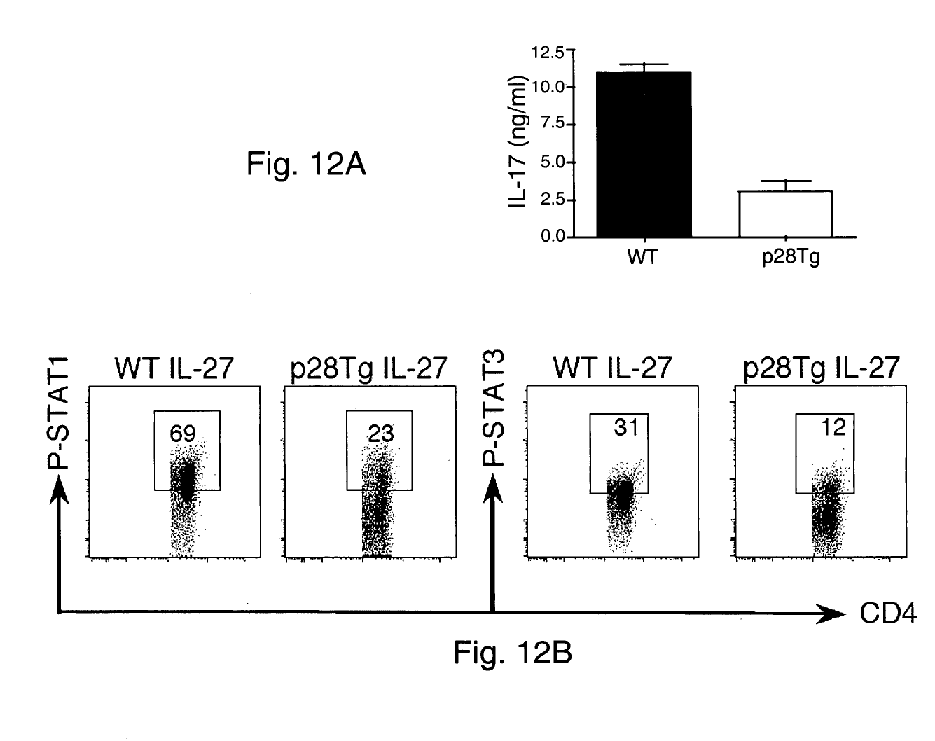

CD4⁺ T cells from wild-type and p28Tg mice were cultured under Th17-inducing conditions and IL-17 production was subsequently measured. In these studies, the production of IL-17 was limited by the transgenic expression of p28 compared to the littermate control (FIG. 9c and FIG. 12a). Furthermore, the p28Tg CD4⁺ T cells produced lower amounts of IL-10 in response to the combination of TGF-f3 plus IL-6, or IL-27 alone (FIG. 9d). Evaluation of the potential for p28 secreted by the transgenic mice to activate cell signaling pathways revealed that it was unable to phosphorylate STAT1 or STAT3 in purified CD4⁺ T cells (FIG. 9e and FIG. 12b). However, like the exogenously added recombinant p28, the ability of IL-6 and IL-27 to phosphorylate STAT1 and STAT3 in CD4⁺ lymphocytes was impaired by the transgenic expression of p28 (FIG. 9e). Moreover, when wild-type and p28Tg T cells were cultured for three days under Th17 polarizing conditions, in order to induce their receptor expression, followed by a two hour incubation on ice prior to stimulation with IL-11 or IL-23, the p28Tg T cells displayed reduced phosphorylation of STAT3 in response to these cytokines compared to wild-type T cells. Together, these studies indicate that the p28 produced by the transgenic cells was able to efficiently antagonize the activity of IL-6 and IL-27 in vitro similar to what was observed for recombinant p28 (FIG. 7c, d). Additionally, the transgenic production of p28 was able to inhibit STAT phosphorylation by the gp130 signaling cytokines IL-6, IL-11 and IL-27 as well as another Type I cytokine IL-23, which does not signal through gp130, but does signal through the IL-12101 chain that shares structural homology to gp130.

Intracellular staining for phosphorylated STAT1 and STAT3 was performed as follows: T cells were purified from C57BL/6 mice with a column (R&D Systems). Purified T cells (1×10⁶) were incubated with IL-6 (10 ng/ml), IL-27 (50 ng/ml), or IL-6 hyperkine (20 ng/ml) for 15 min. Additionally, T cells were pre-incubated with rp28 (100 ng/ml) for 2 h prior to stimulation with IL-6, IL-27, or IL-6 hyperkine. Cells were then fixed for 10 min at 37° C. with 2% (wt/vol) paraformaldehyde. After being fixed, cells were made permeable for 30 min on ice with 90% (vol/vol) methanol, then were stained for phoshorylated STAT1, STAT3, CD4 and CD8. Antibodies to phoshorylated tyrosine residues of STAT1 (clone 4a) and STAT3 (clone 4/P-STAT3) were from BD Pharmingen.

### Overexpression of p28 in Vivo Prevents the Formation of Germinal Centers Following Immunization

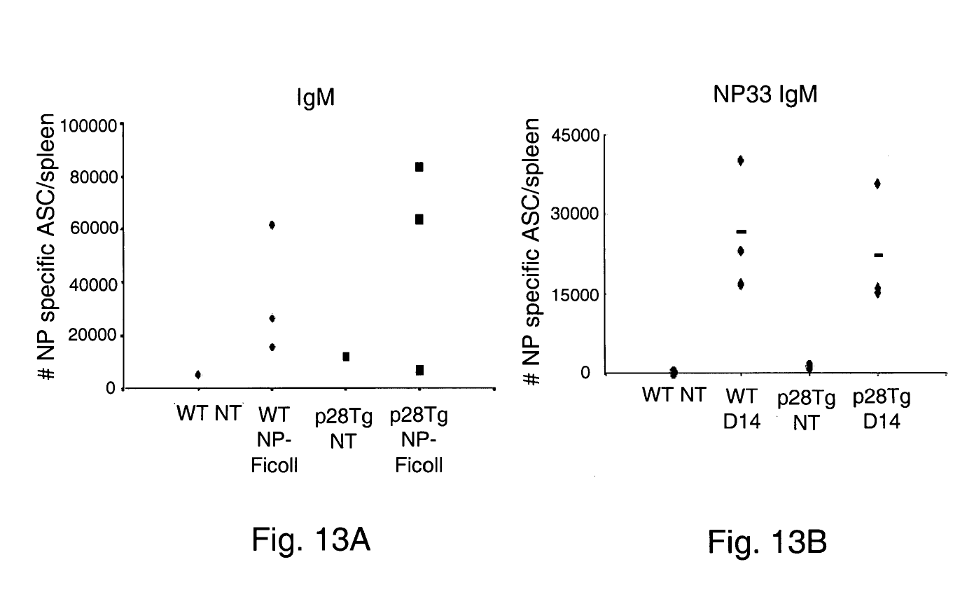

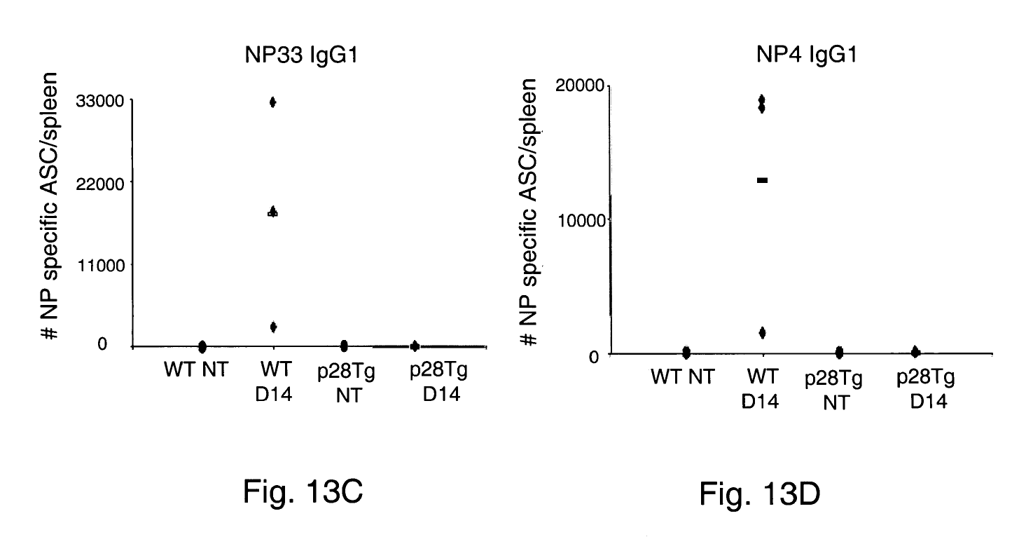

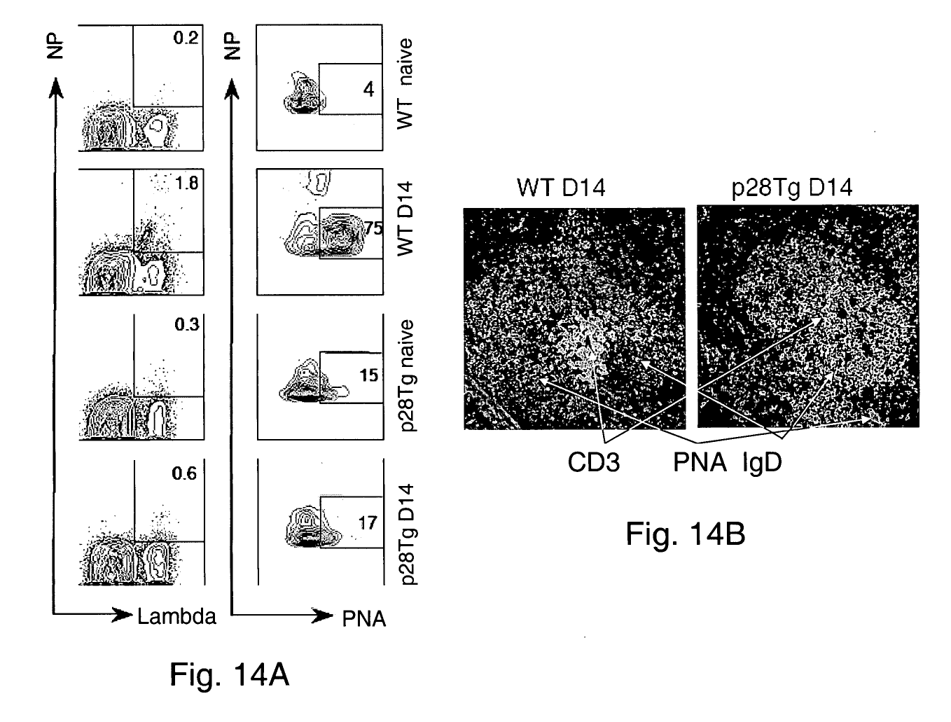

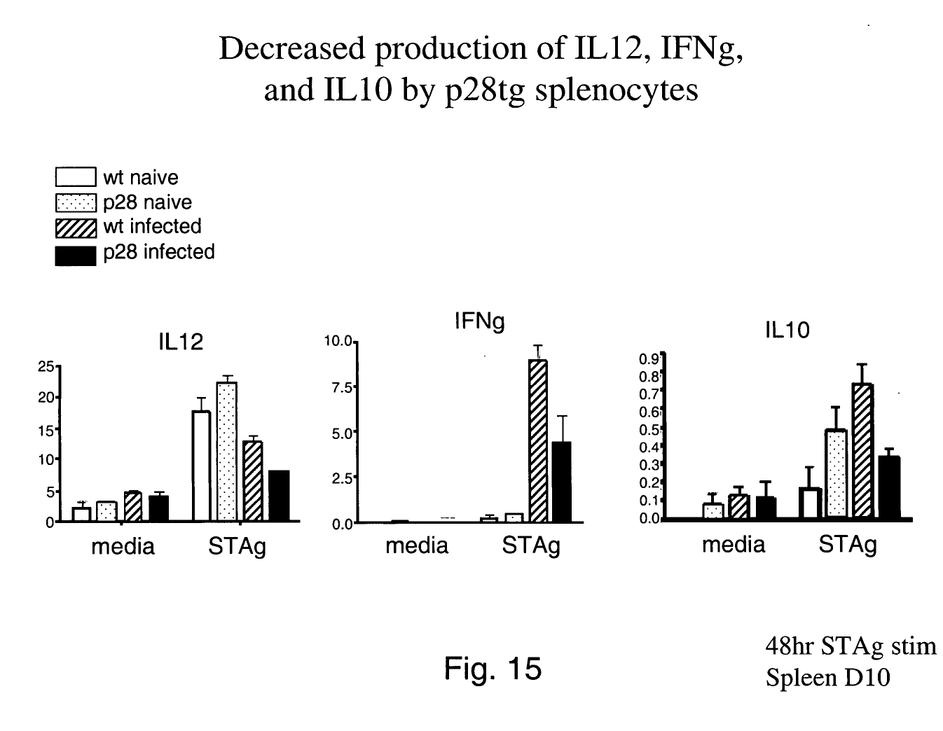

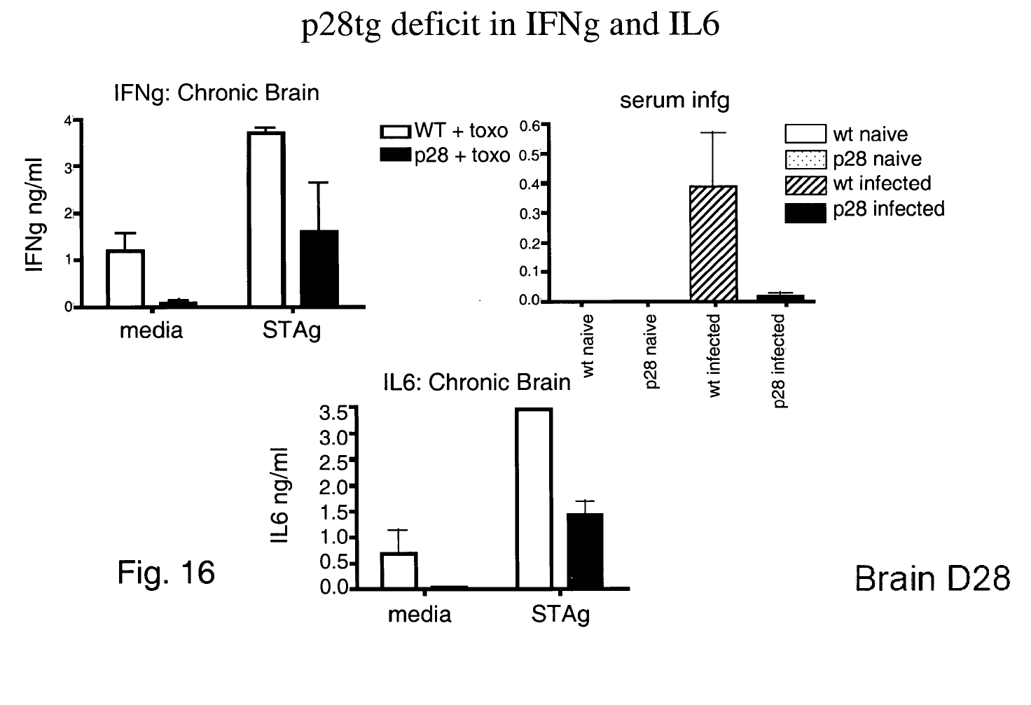

In addition to IL-6 and IL-27, the gp130 subunit is shared by a number of other cytokines including IL-11, leukemia inhibitory factor (LIF), ciliary neurotrophic factor (CNTF) and cardiotrophin-1 (CT-1) (Kishimoto et al. (1995) Interleukin-6 family of cytokines and gp130. Blood 86: 1243-1254). Based on their shared usage of gp130 these cytokines display similar functional activity including promoting neuron survival and/or differentiation in vitro (Kishimoto (1989) “The biology of interleukin-6.” Blood 74: 1-10), induction of the acute phase protein serum amyloid A (Benigni et al. (1996) “Six different cytokines that share GP130 as a receptor subunit, induce serum amyloid A and potentiate the induction of interleukin-6 and the activation of the hypothalamus-pituitary-adrenal axis by interleukin-1.” Blood 87: 1851-1854), food intake reduction (Ulich et al. (1995) “Hematologic effects of stem cell factor (SCF) and leukemia inhibitory factor (LIF) in vivo: LIF-induced thrombocytosis in SCF-primed mice.” Eur J Haematol 54: 217-225) and stimulation of hematopoiesis (Hangoc et al. (1993) “In vivo effects of recombinant interleukin-11 on myelopoiesis in mice.” Blood 81: 965-972; Metcalf et al. (1990) “Effects of injected leukemia inhibitory factor on hematopoietic and other tissues in mice.” Blood 76: 50-56). Furthermore, a number of gp130 signaling cytokines have been shown to have effects on the adaptive immune response including B cell development and antibody production (Muraguchi et al. (1981) “T cell-replacing factor—(TRF) induced IgG secretion in a human B blastoid cell line and demonstration of acceptors for TRF.” J Immunol 127: 412-416; Senaldi et al. (2002) “Regulatory effects of novel neurotrophin-1/b cell-stimulating factor-3 (cardiotrophin-like cytokine) on B cell function.” J Immunol 168: 5690-5698; Senaldi et al. (1999) “Novel neurotrophin-1/B cell-stimulating factor-3: a cytokine of the IL-6 family.” Proc Natl Acad Sci USA 96: 11458-11463). Therefore, based on the involvement of gp130 signaling cytokines in antibody production, and the finding that p28Tg mice have decreased steady-state numbers of IgG antibody-secreting cells we sought to determine if transgenic expression of p28 affects the ability of gp130 signaling to regulate antibody production. Consequently, wild-type and p28Tg mice were immunized intraperitoneally with either the T-independent (TI) antigen 2,4 dinitrophenol-conjugated to Ficoll (NP-Ficoll) in saline or the T-dependent (TD) antigen NP-conjugated to chicken gamma globulin (NP-CGG) in alum followed by measurement of antigen-specific antibody-secreting cells by ELISPOT. Examination of the IgM response to NP-Ficoll at day 5 post-immunization indicated that there was no significant difference in the number of NP-specific IgM secreting cells detected in the spleen of wild-type and p28Tg mice (FIG. 13a). As the antibody response generated against TI antigens occurs in the absence of germinal center (GC) formation this result suggests that the transgenic expression of p28 does not affect the ability of B cells to mount an extrafollicular antibody response. Additionally, there was no difference in the number of NP-specific IgM producing cells derived from the spleen of wild-type and p28Tg mice following immunization with NP-CGG at day 7 or 14 (FIG. 13b). However, while cells isolated from the spleen and bone marrow of wild-type mice were able to effectively generate a NP33-specific IgG1 response no antigen-specific IgG1 secreting cells were detected at either site in the p28Tg mice on day 7 or 14 post-immunization (FIG. 13c). Furthermore, only the B cells from the wild-type mice at day 7 and 14 had undergone affinity maturation as no NP4-specific IgG1 secreting cells were detected in the spleen or bone marrow of p28Tg mice (FIG. 13d). Moreover, when the NP₃₃-specific IgG₁ and NP₄-specific IgG₁ response was measured in the bone marrow at day 14 post-immunization no antigen-specific IgG₁ secreting cells were observed in the p28Tg mice. Consequently, the overall NP-specific antibody response was deficient in the bone marrow of the transgenic mice as opposed to wild-type mice, which had a sufficient anti-NP response. Since IgG₁ production in response to a TD antigen requires the formation of GC reactions the absence of IgG₁ secreting cells in the spleen and bone marrow of p28Tg mice suggests that the transgenic expression of p28 causes a defect in GC formation. Visualization of GC reactions at day 14 post-immunization revealed that the wild-type mice were able to form distinct GCs in the spleen as assessed by the GC marker peanut agglutinin (PNA) and CD3 staining, while the p28Tg mice failed to generate GCs at all, with the majority of the PNA B cells primarily residing outside of the follicle. Also, as the NP response in C57BL/6 mice is idiotypically restricted (Jack, R. S., T. Imanishi-Kari, and K. Rajewsky. 1977. Idiotypic analysis of the response of C57BL/6 mice to the (4-hydroxy-3-nitrophenyl)acetyl group. Eur J Immunol 7:559-565; Reth, M., G. J. Hammerling, and K. Rajewsky. 1978. Analysis of the repertoire of anti-NP antibodies in C57BL/6 mice by cell fusion. I. Characterization of antibody families in the primary and hyperimmune response. Eur J Immunol 8:393-400) and is characterized by the use of the X1 light chain, λ can be used as a marker for NP specificity. Therefore, we sought to confirm the ELISPOT and immunohistochemistry results by determining the number NP⁺λ⁺PNA⁺ GC B cells in the spleen of wild-type and p28Tg mice at day 14 post-NP-CGG-immunization. While naïve wild-type and p28Tg mice showed very few, if any, NP⁺λ⁺PNA⁺ GC B cells in the spleen there was an expansion of this population in the wild-type mice following immunization with NP-CGG. However, the immunized p28Tg mice displayed virtually no expansion of NP⁺λ⁺PNA⁺ GC B cells beyond that seen in the naïve p28Tg mice FIG. 14). Furthermore, immunization with another TD antigen, keyhole limpet hemocyanin (KLH) in complete Freund's adjuvant, yielded similar results in that p28Tg mice were unable to form proper GC reactions in the spleen and thus, no antigen-specific IgG₁ or IgG₂ₐ antibodies were produced by these mice compared to their wild-type littermates, which showed an expansion in PNA⁺ GC B cells in the spleen that were capable of class switching in an antigen specific manner. Given that the p28Tg mice have fewer IgG1 secreting cells in their spleen this suggested that these mice may have a defect in visualization of the germinal center (GC) reactions in these mice at day 14 post-immunization revealed that the wild-type mice were able to form distinct GCs in the spleen as assessed by the GC marker peanut agglutinin (PNA) and CD3 staining, while the p28Tg mice failed to generate GCs at all with the majority of the PNA+ B cells primarily residing outside of the follicle (FIG. 13b). Together, these data indicate that the p28Tg mice are unable to form GC reactions, which are necessary for B cell class switching and affinity maturation to occur in response to immunization with the T cell dependent antigen NP-CGG and KLH.

The immunohistochemcal staining was performed as follows: Organs were embedded in OCT (Electron Microscopy Services, Hatfield, Pa.) and flash frozen. 5 μm sections were cut on a Leica CM3050 Cryostat (Bannockburn, Ill.). Sections were fixed in ice-cold 75% acetone/25% ethanol for 10 minutes. Staining was performed with antibodies specific for B220 (2.5 μg/ml, BD Bioscience) and CD3 (5 μg/Abcam), along anti-rat Cy3 (Jackson Immunoresearch) and anti-rabbit Alexa 488 (Invitrogen) secondary reagents, respectively. Images were captured using standard fluorescence microscopy using a Nikon Eclipse E600 microscope (Melville, N.Y.) equipped a Photometrics Cool Snap EZ CCD camera (Tucson, Ariz.). Nikon NIS Elements software was used to capture and overlay images.

Although known to form the heterodimeric protein IL-27 it is conceivable that the four helix-bundle cytokine p28 and the receptor-like EBI3 proteins may dimerize with other partners, or may have individual activities of their own. There are cases in which EBI3 and p28 have been found not to be expressed by the same cells (Devergne et al. (1996) “A novel interleukin-12 p40-related protein induced by latent Epstein-Barr virus infection in B lymphocytes.” J Virol 70: 1143-1153; Maaser et al. (2004) “Expression of Epstein-Barr virus-induced gene 3 and other interleukin-12-related molecules by human intestinal epithelium.” Immunology 112: 437-445; Collison et al. (2007) “The inhibitory cytokine IL-35 contributes to regulatory T-cell function.” Nature 450: 566-569). While transcription of each subunit has been found to be induced by similar stimuli there are some factors that preferentially induce the transcription of one protein over the other (Pflanz et al. (2002) “IL-27, a heterodimeric cytokine composed of EBI3 and p28 protein, induces proliferation of naive CD4(+) T cells.” Immunity 16: 779-790; Hibbert et al. (2003) “IL-27 and IFN-alpha signal via Stat 1 and Stat3 and induce T-Bet and IL-12Rbeta2 in naive T cells.” J Interferon Cytokine Res 23: 513-522; Sonobe et al. (2005) “Production of IL-27 and other IL-12 family cytokines by microglia and their subpopulations.” Brain Res 1040: 202-207). Additionally, the kinetics of p28 and EBI3 expression have been reported to differ following activation of monocyte-derived dendritic cells in that p28 expression peaks early following activation (6-12 h), while EBI3 expression is sustained and peaks later (28-24 h) (Schnurr et al. (2005) “Extracellular nucleotide signaling by P2 receptors inhibits IL-12 and enhances IL-23 expression in human dendritic cells: a novel role for the cAMP pathway.” Blood 105: 1582-1589). Recently, the p35 subunit of IL-12 has been reported to associate with EBI3 to form a heterodimeric cytokine termed IL-35, which is produced primarily by regulatory T cells, and whose function remains unresolved (Collison et al. (2007) “The inhibitory cytokine IL-35 contributes to regulatory T-cell function.” Nature 450: 566-569; Niedbala et al. (2007) “IL-35 is a novel cytokine with therapeutic effects against collagen-induced arthritis through the expansion of regulatory T cells and suppression of Th17 cells.” Eur J Immunol 37: 3021-3029). It has also been described that the p19 subunit of IL-23 binds to EBI3, and that p28 binds a receptor-like protein CLF; however, whether these complexes form in vivo is still unclear (Kastelein et al. (2007) “Discovery and biology of IL-23 and IL-27: related but functionally distinct regulators of inflammation.” Annu Rev Immunol 25: 221-242, Crabe et al. (2008) “A new composite cytokine formed by the interleukin-27 subunit P28 and soluble receptor CLF activating human natural killer cells.” Cytokine 43: 263).

Thus, in light of this idea our laboratory has shown that recombinant murine p28 has biological activity (Stumhofer et al. (2006) “Interleukin 27 negatively regulates the development of interleukin 17-producing T helper cells during chronic inflammation of the central nervous system.” Nat Immunol 7: 937-945) in that it could limit Th17 development in vitro. In addition, this study has confirmed that the activity of p28 does not require the presence of EBI3, nor does it require a cell-signaling event. Furthermore, p28 secretion could be detected by activated antigen presenting cells and the serum of mice in the presence or absence of EBI3, thus, confirming the findings by Pflanz et al. (Pflanz et al. (2002) “IL-27, a heterodimeric cytokine composed of EBI3 and p28 protein, induces proliferation of naive CD4(+) T cells.” Immunity 16: 779-790). Also, p28 was shown to inhibit and not induce the expression of IL-10, and thus, it does not fully recapitulate the activity of IL-27 indicating that the function of p28 is unique to this protein. Moreover, using p28 transgenic mice that have B and T lymphocytes that over-express the p28 gene we confirmed our in vitro findings with recombinant murine p28 by showing that CD4⁺ T cells from the p28Tg mice produce less IL-17 and IL-10 in response to TGF-β and IL-6. Also, the level of STAT1 and STAT3 phosphorylation in the p28Tg T cells was diminished in response to IL-6 and IL-27. Therefore, based on these findings we have concluded that p28 can be used to antagonize or inhibit of IL-6 and IL-27 mediated signaling.

What is claimed is:

1. A method of treating or preventing an autoimmune disease, the method comprising administering p28 to a subject at risk for the autoimmune disease or to a subject who has the autoimmune disease.

2. The method of claim 1, wherein the autoimmune disease is mediated by B cell production of antibodies.

3. The method of claim 1, wherein the autoimmune disease is Systemic lupus erythematosus (SLE), Autoimmune hepatitis, Bullous pemphigoid, Celiac disease, Guillain-Barré syndrome (GBS), Goodpasture's syndrome, Multiple sclerosis associated with the presence of autoantibodies to Myelin basic protein (MBP) and Myelin oligodendrocyte glycoprotein (MOG), Pemphigus Vulgaris, Primary biliary cirrhosis, Rheumatoid arthritis associated with rheumatoid factor, Scleroderma, or Wegener's granulomatosis.

4. The method of claim 1, further comprising administering an additional antagonist of T and B cell interactions.

5. The method of claim 4, wherein the additional antagonist blocks an interaction of ICOS-ICOS-L, IL-21, or CD40-CD40L.

6. The method of claim 1, wherein said p28 is a p28 variant.

7. The method of claim 6, wherein the modified p28 has an increased half-life.

8. The method of claim 1, wherein the subject is human.

9. A method of enhancing an IL-6 mediated immune response in a subject, the method comprising administering an inhibitor of p28 to the subject.

10. The method of claim 9, wherein the subject is a recipient of a vaccination for infectious disease or an immune-mediated cancer therapy.

11. The method of claim 9, wherein the subject is human.

12. A method of treating or preventing a gp130-associated cancer, the method comprising:
a. identifying a subject who has or is predisposed to develop the gp130 associated cancer; and,
b. administering p28 to the subject.

13. The method of claim 12, further comprising administering an additional gp130 antagonist or cytokine antagonist.

14. A method of suppressing an immune response in a subject, the method comprising administering IL-1Ra and p28 to the subject.

15. A method of suppressing an immune response, the method comprising administering a combination of at least two inhibitors of Th17 differentiation.

16. The method of claim 15, wherein one of the at least two inhibitors of Th17 differentiation is p28.

17. The method of claim 15, wherein one of the at least two inhibitors of Th17 differentiation is an IL-1 antagonist, an IL-21 antagonist, a TNF antagonist, or an IL-23 antagonist or CTLA4-Ig.

18. A method of limiting transplant rejection, the method comprising, administering p28 to a transplant recipient.
